(19) United States
(12) Patent Application Publication
(10) Pub. No.: US 2013/0143253 A1
(43) Pub. Date: Jun. 6, 2013

(54) METHOD FOR QUANTITATIVE MEASUREMENT OF GASTRIC ACIDITY USING 13C CARBONATE SALT

(71) Applicants: Makoto Inada, Osaka-shi (JP); Jun-ichi Kunizaki, Osaka-shi (JP); Kazuki Tobita, Osaka-shi (JP); Suguru Akamatsu, Osaka-shi (JP); Shinji Iizuka, Osaka-shi (JP)

(72) Inventors: Makoto Inada, Osaka-shi (JP); Jun-ichi Kunizaki, Osaka-shi (JP); Kazuki Tobita, Osaka-shi (JP); Suguru Akamatsu, Osaka-shi (JP); Shinji Iizuka, Osaka-shi (JP)

(21) Appl. No.: 13/817,349

(22) PCT Filed: Aug. 18, 2011
(86) PCT No.: PCT/JP2011/068714

§ 371 Date: Feb. 15, 2013

(30) Foreign Application Priority Data
Aug. 19, 2010 (JP) 2010-184486

(51) Int. Cl. G01N 33/497; C12Q 1/26

(52) U.S. Cl. CPC G01N 33/497; C12Q 1/26
USPC 435/25; 436/133

(57) ABSTRACT

The present invention provides a method for measuring the gastric acidity of a mammal using a ¹³C-labeled carbonate compound. Specifically, the present invention relates to a method for measuring the gastric acidity of a mammal including the following steps:
(1) using, as a test sample, expired air of a mammalian subject excreted at any point in time within 30 minutes after oral administration of a predetermined amount of a ¹³C-labeled carbonate compound, measuring behavior of ¹³CO₂ in the expired air;
(2) comparing the behavior of ¹³CO₂ (measured ¹³CO₂ behavior) obtained in step (1) with the behavior of corresponding ¹³CO₂ (reference ¹³CO₂ behavior) that has been obtained beforehand in a control mammal; and
(3) determining the gastric acidity of the mammalian subject based on a difference between the reference ¹³CO₂ behavior and the measured ¹³CO₂ behavior obtained above.

### TECHNICAL FIELD

The present invention relates to a method for measuring the gastric acidity of a mammal using a ¹³C-labeled carbonate compound. More specifically, the present invention relates to a method for non-invasively and quantitatively measuring the gastric acidity of a mammal using expired air excreted after the administration of a ¹³C-labeled carbonate compound.

The present invention also relates to a method for diagnosing a disease relating to gastric acid secretion by measuring gastric acidity tendency (hyperacidity, normal, hypoacidity, or anacidity), and a method for measuring the effect of a drug that has the action of suppressing gastric acid (hereinafter referred to as a “gastric acid inhibitor”).

Further, the present invention relates to, from another perspective as examples of the application of the above methods, a method for evaluating the enzyme activity (metabolic capacity) of CYP2C19 alone or both CYP2C13 and CYP3A4 in a subject; and a method for evaluating the effect of a drug metabolized by CYP2C19 alone or both CYP2C19 and CYP3A4 on a subject, and the susceptibility of a subject to the drug.

### BACKGROUND ART

A large number of drugs are synthesized in the form of organic acids or organic bases. It is known that some of these organic acids and organic bases are influenced by gastric acidity, causing large changes in bioavailability; as a result, they do not produce the expected pharmacological effects, or cause unexpected and severe side effects. Further, in today's aging society, the number of patients with hypoacidity or anacidity is said to be rapidly increasing.

In the case of such patients, it is believed that measuring the tendency (hyperacidity, normal, hypoacidity, anacidity, or the hike) of gastric acidity (HCl concentration×amount of gastric juice) before medication provides very useful information for selecting a drug, and predicting the therapeutic and side effects of the drug. The advent of gastric acid secretion inhibitors, such as H₂-antagonists and proton pump inhibitors (PPIs), has greatly contributed to the treatment of gastric and duodenal ulcers. However, recrudescence or recurrence (in particular, recurrence of reflux esophagitis) after treatment has become a problem, in recent years; therefore, medicinal treatment methods for gastric and duodenal ulcers, including revisions of treatment, methods, are attracting attention as a subject to be examined in the medical field. Treatment with a gastric acid secretion inhibitor suppresses gastric acid secretion, and its therapeutic effect can be evaluated by measuring basic gastric acid secretion. The recurrence of reflux esophagitis is a rebound phenomenon caused when the administration of a gastric, acid secretion inhibitor is discontinued, and is presumed to be predictable to some extent by measuring gastric acid output.

Thus, the measurement of gastric acidity presumably makes it possible to predict the therapeutic and side effects of a drug to some extent. Further, since the gastric acidity tendency has become clear in some diseases (e.g., gastric ulcer, duodenal ulcer, gastric cancer, chronic gastritis, liver/biliary tract/pancreatic disease, pernicious anemia, vitamin B complex deficiency disease, pyloric stenosis, Zollinger-Ellison syndrome, and the like), it is believed that the measurement of gastric acidity will find wide application in diagnosing diseases.

Known methods for measuring gastric acid output include a method comprising sucking acid from the stomach through a naso-gastric tube, while giving a stimulus to promote secretion. However, this method is not practical since it is invasive, and imposes physical and mental strain on a subject. Proposed as a non-invasive method is a method comprising orally administering a large quantity of a water-insoluble carbonate containing an isotope to a subject, and measuring the amount of gastric secretion from the content of the isotope in carbon dioxide excreted in expired air (Patent Literature 1). However, this method measures the content of the isotope in carbon dioxide produced as a result of complete neutralization of gastric acidity; accordingly, a subject must be given an excess quantity of the carbonate relative to gastric acid volume. Moreover, before collecting expired air, it is necessary to wait for at least 60 minutes (preferably at least 150 minutes), which is the time required for the gastric acid to be completely neutralized with the administered carbonate plus the time for the isotope content in carbon dioxide in the expired air to be stabilised; accordingly, the measurement problematically requires time.

Also proposed is a method comprising orally administering an isotope-containing composition to a subject, and non-invasively measuring gastric pH from the amount of the isotope in carbon dioxide excreted in expired airy as in the above method (Patent Literature 2). However, this method is based on the finding that when a composition containing an isotope-labeled compound is covered with a pH-dependent soluble base, such as an enteric base or a gastro-soluble base, the behavior of the isotope in carbon dioxide excreted from the body changes according to the gastric pH, and there is a constant relation between the gastric pH and the excretion behavior of the labeled compound. Thus, this method requires the use of an administration preparation covered with a pH-dependent soluble base.

### CITATION LIST

### Patent Literature

PTL 1: Japanese Unexamined Patent Publication No. 2009-515139
PTL 2: WO01/97863A1

### SUMMARY OF INVENTION

### Technical Problem

An object of the present invention is to provide a method for quantitatively measuring the gastric, acidity of a mammal, including a human, in a non-invasive and simple manner, using a ¹³C-labeled carbonate compound.

Another object of the present invention is to provide a method for diagnosing a disease relating to gastric acid secretion by measuring gastric acidity tendency (hyperacidity, normal, hypoacidity, or anacidity), and a method for measuring the effect of a gastric acid reducer (e.g., gastric, acid secretion inhibitors, such as proton pump inhibitors and H₂ blockers; and drugs for neutralising gastric acid, such as antacids). An additional object of the present invention is to provide, as examples of the application of the above methods, a method for evaluating the enzyme activity (metabolic capacity) of CYP2C19 alone or both CYP2C19 and CYP3A4 in a subject; and a method for evaluating the effect of a drug metabolized by CYP2C19 alone or both CYP2C19 and CYP3A4 on a subject, and the susceptibility of a subject to the drug.

### Solution to Problem

To solve the above problems, the present inventors analyzed ¹³CO₂ excretion behavior in expired air obtained after orally administering a ¹³C-labeled carbonate compound to mammals, and found that, regardless of the degree of gastric acidity of the mammals, there is a linear correlation passing through the origin between the dose of the ¹³C-labeled carbonate compound and Δ¹³C(‰) at a point in time for collecting the expired air. Here, “Δ¹³C(‰)” means the difference [Δ¹³C(‰)=(δ¹³C)ₜ−(δ¹³C)₀] between the ratio (“(δ¹³C)ₜ”) of ¹³CO₂ amount to ¹²CO₂ amount in the expired air at a point, in time for collecting the expired air (t), and the ratio (“(δ¹³C)₀”) of ¹³CO₂ amount to ¹²CO₂ amount in the expired air before the administration of the ¹³C-labeled carbonate compound. In addition, “dose” means the amount of the ¹³C-labeled carbonate compound administered to a mammalian subject; and encompasses both the dose (number of moles, weight) of the ¹³C-labeled carbonate compound administered to 1 body of a mammalian subject, and the dose (number of moles, weight) of the ¹³C-labeled carbonate compound administered per kg body weight of a mammalian subject.

The present inventors also found that when the dose exceeds a certain amount, the Δ¹³C(‰) curve versus the dose of the ¹³C-labeled carbonate compound reaches a nearly constant value (Δ¹³C(‰) plateau value) according to the degree of gastric acidity of mammals; that the dose at which the Δ¹³C(‰) reaches a plateau (corresponding to the above “certain amount”) varies depending on the gastric acidity of the mammals; and that there is a corresponding relation between the dose and the gastric acidity. Based on these findings, the present inventors confirmed that the gastric acidity of each subject can be measured and evaluated using the Δ¹³C(‰) plateau value as an index. As described above, regardless of the degree of gastric acidity of mammals, there is a linear correlation passing through the origin between the dose of the ¹³C-labeled carbonate compound and the Δ¹³C(‰) until the Δ¹³C(‰) reaches a plateau.

Further, the present inventors confirmed that, as in the case of the above Δ¹³C(‰), when the dose exceeds a certain amount, the “area under the Δ¹³C(‰)-time curve” (AUC) versus the dose of the ¹³C-labeled carbonate compound reaches a nearly constant value (AUC plateau value; according to the degree of gastric acidity of mammals; that the dose at which the AUC reaches a plateau (corresponding to the above “certain amount”) varies depending on the gastric acidity of the mammals, and there is a corresponding relation between the dose and the gastric acidity; and that, as in the case of the Δ¹³C(‰), the gastric acidity of each subject can be measured and evaluated using the AUC plateau value as an index. Furthermore, as in the case of the Δ¹³C(‰), regardless of the degree of gastric acidity of mammals, there is a linear correlation passing through the origin between the dose of the ¹³C-labeled carbonate compound and the AUC until the AUC reaches a plateau.

As described above, the method of the present invention makes it possible to quantitatively measure and evaluate the gastric acidity of a subject non-invasively in a short period of time, without restraining the subject for a long period of time, by using the Δ¹³C(‰) as an index.

The present invention was accomplished based on these findings, and includes the following embodiments.

### (I) Method for Measuring Gastric Acidity of a Mammal

(I-1) A method for measuring gastric acidity of a mammal comprising the steps of:

(1) using, as a test sample, expired air of a mammalian subject excreted at any point in time within 30 minutes after oral administration of a predetermined amount of a ¹³C-labeled carbonate compound, measuring behavior of ¹³CO₂ excreted in the expired air;

(2) comparing the behavior of ¹³CO₂ (measured ¹³CO₂ behavior) obtained in step (1) with the behavior of corresponding ¹³CO₂ (reference ¹³CO₂ behavior) that has been obtained beforehand in a control mammal; and

(3) determining gastric acidity of the mammalian subject based on a difference between the reference ¹³CO₂ behavior and the measured ¹³CO₂ behavior obtained above.

(I-2) The method according to Item (I-1), wherein the behavior of ¹³CO₂ is Δ¹³C(‰)ₜ (t is an expired air collection time, within 30 minutes) obtained from, expired air of a mammalian subject collected at any point in time within 30 minutes after oral administration of a ¹³C-labeled carbonate compound.
(I-3) The method according to Item (I-1) or (I-2), wherein the predetermined amount is 10 mg to 5 g.
(I-4) The method according to any one of Items (I-1) to (I-3), wherein the ¹³C-labeled carbonate compound is at least one carbonate compound selected from the group consisting of alkali metal carbonates, alkaline earth metal carbonates, ammonium carbonate, alkali metal hydrogencarbonates, and ammonium hydrogencarbonate.
(I-5) The method according to any one of Items (I-1) to (I-3), wherein the ¹³C-labeled carbonate compound is at least one carbonate compound selected from the group consisting of sodium carbonate, potassium carbonate, calcium carbonate, magnesium carbonate, barium carbonate, ammonium carbonate, potassium hydrogen-carbonate, sodium hydrogencarbonate, and ammonium, hydrogencarbonate.
(I-6) The method according to any one of Items (I-1) to (I-5), wherein the expired air used as a test sample is expired air of a mammalian subject excreted at any point in time within 20 minutes, preferably 15 minutes after oral administration of a ¹³C-labeled carbonate compound.
(I-7) The method according to any one of Items (I-2) to (I-6), wherein the control mammal used in comparing step (2) is a mammal having normal gastric acidity, and de termination step (3) is a step of determining that the gastric acidity or the mammalian subject is the same as or higher than the gastric acidity of the control mammal when the measured ¹³CO₂ behavior and the reference ¹³CO₂ behavior are the same, or that the gastric acidity of the mammalian subject is lower than the gastric acidity of the control mammal when the measured ¹³CO₂ behavior is lower than the reference ¹³CO₂ behavior.

### (II) Method for Measuring an Effect, of a Gastric Acid Reducer

(II-1) A method for measuring an effect of a gastric acid reducer on a mammal, the method comprising the following steps (1) to (4):

(1) using, as a test sample, expired air of a mammalian subject excreted at arty point in time within 30 minutes after oral administration of a predetermined amount of a ¹³C-labeled carbonate compound, the oral administration being performed after administration of a gastric acid reducer, measuring behavior of ¹³CO₂ excreted in the expired air,

(2) comparing the behavior of ¹³CO₂ (measured ¹³CO₂ behavior) obtained in step (1) with the behavior of corresponding ¹³CO₂ (reference ¹³CO₂ behavior) measured in a mammal (control mammal) to which a predetermined amount of a ¹³C-labeled carbonate compound has been orally administered beforehand without administering the gastric acid reducer;

(3) determining gastric acidity of the mammalian subject based on a difference between the reference ¹³CO₂ behavior and the measured ¹³CO₂ behavior obtained above; and

(4) determining the effect of the gastric acid reducer on the mammalian subject using the gastric acidity of the mammalian subject obtained above as an index.

(II-2) The method according to Item (II-1), wherein the behavior of ¹³CO₂ is Δ¹³C(‰)ₜ (t is an expired air collection time, within 30 minutes) obtained from expired air of a mammalian subject collected at any point in time within 30 minutes after oral administration of a ¹³C-labeled carbonate compound.
(II-3) The method, according to Item (II-1) or (II-2), wherein the predetermined amount is 10 mg to 5 g.
(II-4) The method according to any one of Items (II-1) to (II-3), wherein the ¹³C-labeled carbonate compound is at least one carbonate compound selected from the group consisting of alkali metal carbonates, alkaline earth metal carbonates, ammonium carbonate, alkali metal hydrogencarbonates, and ammonium hydrogencarbonate.
(II-5) The method according to any one of Items (II-1) to (II-3, wherein the ¹³C-labeled carbonate compound is at least, one carbonate compound selected from the group consisting of sodium carbonate, potassium carbonate, calcium carbonate, magnesium carbonate, barium carbonate, ammonium carbonate, potassium hydrogencarbonate, sodium hydrogencarbonate, and ammonium hydrogencarbonate.
(II-6) The method according to any one of Items (II-1) to (II-5), wherein the expired air used as a test sample is expired air of a mammalian subject excreted at any point in time within 20 minutes, preferably 15 minutes after oral administration of a ¹³C-labeled carbonate compound.
(II-7) The method according to any one of Items (II-1) to (II-6), wherein step (3) is a step of determining that the gastric acidity of the mammalian subject is the same as or higher than the gastric acidity of the control mammal when the measured ¹³CO₂ behavior and the reference ¹³CO₂ behavior are the same, or that the gastric acidity of the mammalian subject is lower than the gastric acidity of the control mammal when the measured ¹³CO₂ behavior is lower than the reference ¹³CO₂ behavior.
(II-8) The method according to Item (II-7), wherein step (4) is a step of determining that the administered gastric acid reducer has no effect on the mammalian subject when the gastric: acidity of the mammalian subject measured in step (3) is the same as or higher than the gastric acidity of the control mammal; or that the administered gastric acid reducer has an effect on the mammalian subject when the gastric acidity of the mammalian subject measured in step (3) is lower than the gastric acidity of the control mammal.
(II-9) The method according to any one of Items (II-1) to (II-9), wherein the gastric: acid reducer is a proton pump inhibitor, an H₂ blocker, or an antacid.
(II-10) The method, according to Item (II-9) wherein the proton pump inhibitor is at least one member selected from the group consisting of omeprazole, lansoprazole, pantoprazole, rabeprazole, and esomeprazole; the H₂ blocker is at least one member selected from the group consisting of ranitidine, cimetidine, famotidine, nizatidine, lafutidine, and roxatidine acetate hydrochloride; and the antacid is at least one member selected from the group consisting of magnesium hydroxide, anhydrous dibasic calcium phosphate, precipitated calcium carbonate, sodium hydrogencarbonate, and magnesium oxide.
(III) Method for Evaluating Enzyme Activity (Metabolic Capacity) of CYP2C13 Alone or Both CYP2C19 and CYP3A4 in a Mammalian Subject, Effect of a Drug Metabolized by CYP2C19 Alone or Both CYP2C19 and CYP3A4 on a Mammalian Subject, or/and Susceptibility of a Mammalian Subject to the Drug
(III-1) A method for evaluating enzyme activity (metabolic capacity) of CYP2C19 alone or both CYP2C19 and CYP3A4 in a mammalian subject, effect of a drug metabolized by CYP2C19 alone or both CYP2C19 and CYP3A4 on the mammalian subject, or/and susceptibility of the mammalian subject to the drug, the method comprising the following steps (1) to (4):

(1) using, as a test sample, expired air of a mammalian subject excreted at any point in time within 30 minutes after oral administration of a predetermined amount of a ¹³C-labeled carbonate compound, the oral administration being performed after administration of omeprazole or lansoprazole, measuring behavior of ¹³CO₂ excreted in the expired air,

(2) comparing the behavior of ¹³CO₂ (measured ¹³CO₂ behavior) obtained in step (1) with behavior of corresponding ¹³CO₂ (reference ¹³CO₂ behavior) measured in a mammal (control mammal) to which a predetermined amount of a ¹³C-labeled carbonate compound has been orally administered beforehand without administering omeprazole and lansoprazole;

(3) determining the gastric acidity of the mammalian subject: based on a difference between the reference ¹³CO₂ behavior and the measured ¹³CO₂ behavior obtained above; and

(4) determining the enzyme activity (metabolic capacity) of CYP2C19 alone or both CYP2C19 and CYP3A4 in the mammalian subject, effect of a drug metabolized by CYP2C19 alone or both CYP2C19 and CYP3A4 on the mammalian subject, or/and susceptibility of the mammalian subject to the drug, using the gastric acidity of the mammalian subject obtained above as an index.

(III-2) The method according to Item (III-1), wherein the behavior of ¹³CO₂ is Δ¹³C(‰)ₜ (t is an expired air collection time, within 30 minutes) obtained from expired air of a mammalian subject collected at any point in time within 30 minutes after oral administration of a ¹³C-labeled carbonate compound.
(III-3) The method according to Item (III-1) or (III-2), wherein the predetermined amount is 10 mg to 5 g.
(III-4) The method according to any one of items (III-1) to (III-3), wherein the ¹³C-labeled carbonate compound is at least one carbonate compound selected from the group consisting of alkali metal carbonates, alkaline earth metal carbonates, ammonium carbonate, alkali metal hydrogencarbonates, and ammonium hydrogencarbonate.
(III-5) The method according to any one of Items (III-1) to (III-3), wherein the ¹³C-labeled carbonate compound is at least one carbonate compound selected from the group consisting of sodium carbonate, potassium carbonate, calcium carbonate, magnesium carbonate, barium carbonate, ammonium carbonate, potassium hydrogencarbonate, sodium hydrogencarbonate, and ammonium hydrogencarbonate.
(III-6) The method according to any one of Items (III-1) to (III-5), wherein the expired air used as a test sample is expired air of a mammalian subject excreted at any point in time within 20 minutes, preferably 15 minutes after oral administration of a ¹³C-labeled carbonate compound.
(III-7) The method according to any one of Items (III-1) to (III-6), wherein step (3) is a step of determining that the gastric acidity of the mammalian subject is the same as or higher than the gastric acidity of the control mammal when the measured ¹³CO₂ behavior and the reference ¹³CO₂ behavior are the same, or that the gastric acidity of the mammalian subject is lower than the gastric acidity of the control mammal when the measured ¹³CO₂ is lower than the reference ¹³CO₂.
(III-8) The method according to Item (III-7) comprising as step (4) a step of determining enzyme activity (metabolic capacity) of CYP2C19 alone or both CYP2C19 and CYP3A4 in a mammalian subject, wherein step (4) is a step of determining that the enzyme activity (metabolic capacity) of CYP2C19 alone or both CYP2C19 and CYP3A4 in the mammalian subject is normal or high when step (3) determines that the gastric acidity of the mammalian subject is the same as or higher than the gastric acidity of the control mammal; or that the enzyme activity (metabolic capacity; of CYP2C19 alone or both CYP2C19 and CYP3A4 in the mammalian subject is low when step (3) determines that the gastric acidity of the mammalian subject is lower than the gastric acidity of the control mammal.
(III-9) The method according to Item (III-7) or (III-8) comprising as step (4) a step of determining effect of a drug metabolized by CYP2C19 alone or both CYP2C19 and CYP3A4 on a mammalian subject, wherein

(a) in the case where the drug metabolized by CYP2C19 alone or both CYP2C19 and CYP3A4 shows an effect before being metabolized, step (4) is a step of determining that the effect of the drug on the mammalian subject is low when step (3) determines that the gastric acidity of the mammalian subject is the same as or higher than the gastric acidity of the control mammal, or that the effect, of the drug on the mammalian subject is high when step (3) determines that the gastric acidity of the mammalian subject is lower than the gastric acidity of the control mammal; or

(b) in the case where the drug metabolized by CYP2C19 alone or both CYP2C19 and CYP3A4 shows an effect, by metabolization, step (4) is a step of determining that the effect of the drug on the mammalian subject is high when step (3) determines that the gastric acidity of the mammalian subject, is the same as or higher than the gastric acidity of the control mammal, or that the effect of the drug on the mammalian subject, is low when step (3) determines that the gastric acidity of the mammalian subject is lower than the gastric acidity of the control mammal.

(III-10) The method according to any one of Items (III-7) to (III-9) comprising as step (4) a step of determining susceptibility of a mammalian subject to a drug metabolized by CYP2C19 alone or both CYP2C19 and CYP3A4, wherein

(a) in the case where the drug metabolized by CYP2C19 alone or both CYP2C19 and CYP3A4 shows an effect before being metabolized, step (4) is a step of determining that the susceptibility of the mammalian subject to the drug is high when step (3) determines that the gastric acidity of the mammalian subject is the same as or higher than the gastric acidity of the control mammal, or that the susceptibility of the mammalian subject to the drug is low when step (3) determines that the gastric acidity of the mammalian subject is lower than the gastric acidity of the control mammal; or

(b) in the case where the drug metabolized by CYP2C19 alone or both CYP2C19 and CYP3A4 shows an effect by metabolization, step (4) is a step of determining that the susceptibility of the mammalian subject to the drug is low when step (3) determines that the gastric acidity of the mammalian subject is the same as or higher than the gastric acidity of the control mammal, or that the susceptibility of the mammalian subject to the drug is high when step (3) determines that the gastric acidity of the mammalian subject is lower than the gastric acidity of the control mammal.

(III-11) The method according to Item III-7) or (III-8), wherein the drug metabolized by CYP2C19 alone or both CYP2C19 and CYP3A4 is any one of those selected from the group consisting of diazepam, omeprazole, lansoprazole, propranolol, and clopidogrel.
(III-12) The method according to Item (III-9) or (III-10), wherein the drug that exhibits an effect before being metabolized by CYP2C19 alone or both CYP2C19 and CYP3A4 is any one of those selected from the group consisting of diazepam, omeprazole, lansoprazole, and propranolol.
(III-13) The method according to (III-9) or (III-10), wherein the drug that, exhibits an effect by being metabolized by CYP2C19 alone or both CYP2C19 and CYP3A4 is clopidogrel.

### Advantageous Effects of Invention

The method of the present invention for measuring gastric acidity makes it possible to quantitatively measure gastric acidity in a simple manner by using an expiration test using a ¹³C-labeled carbonate compound as an oral preparation, without placing any mental or physical burden on a subject that is a mammal, including a human.

In addition, the method, of the present invention makes it possible to diagnose a disease relating to gastric acid secretion for a mammalian subject; to measure and evaluate the effect of a drug relating to gastric acid secretion (e.g., gastric acid secretion inhibitors such as proton pump inhibitors and H₂ blockers) or a drug that has the action of neutralizing gastric acid (e.g., antacids) (these drugs are referred to as “gastric acid reducers”); or to easily measure and evaluate the effect of a drug metabolized by CYP2C19 alone or both CYP2C19 and CYP3A4, and the susceptibility to the drug (for example, including deficiencies, etc., in these metabolism enzymes).

For example, in the case where proton pump inhibitors, such as lansoprazole, among drugs relating to gastric acid secretion, are used, the drugs are metabolized by hepatic metabolism enzymes CYP2C19 and CYP3A4. Thus, by measuring the gastric acidity of mammalian subjects after administration of these drugs, the enzyme activity (metabolic capacity) of CYP2C19 and CYP3A4 in the mammalian subjects (reduction or elevation in CYP2C19 and CYP3A4 due to genetic defects or presence of polymorphisms of the hepatic metabolism enzymes) can be measured and evaluated.

Further, from this perspective, the present invention can provide a method for measuring and evaluating the enzyme activity (metabolic capacity) of CYP2C19 alone or both CYP2C19 and CYP3A4 in a mammal, including a human, used as a test subject; and can also provide a method for measuring and evaluating the effect on a mammalian subject of a drug that exhibits its effect before being metabolized by the enzyme or enzymes, or of a drug that exhibits its effect by being metabolized by the enzyme or enzymes (this can also be regarded as the susceptibility of a mammalian subject to these drugs).

### BRIEF DESCRIPTION OF DRAWINGS

FIG. 1 (1) shows the correlation between each dose of the administration preparation and Δ¹³C(‰). FIG. 1 (2) shows the correlation between each dose of the administration preparation and the “area under the Δ¹³C(‰)-time curve” (AUCt). Here, “time” means a time it) from when the administration preparation is administered to when expired air is collected. In the dose up to a certain amount, linear correlations passing through the origin are shown between the dose and the Δ¹³C(‰), and between the dose and the AUCt, regardless of the degree of the gastric acidity of the subjects (mammals). It is also shown that the Δ¹³C(‰) plateau value and the dose thereof at which the Δ¹³C(‰) becomes constant and the AUCt plateau value and the dose thereof at which the AUCt becomes constant vary depending on the degree of the gastric acidity of the subjects (mammals) (a: high gastric acidity, b: normal gastric acidity, and c: low gastric acidity).

FIG. 2 is a graph showing changes over time in Δ¹³C(‰) measured from ¹³CO₂ in expired air after each of the ¹³C—CaCO₃ suspensions at various concentrations (2500, 1000, 500, 200, 100, 20, and 4 μmol/4 mL) was administered to rats in Experimental Example 1. In FIG. 2, the Δ¹³C(‰) [(δ¹³Cₜ)−(δ¹³C₀)] in the expired air is plotted on the ordinate, whereas the expired air collection time (minutes) after the ¹³C—CaCO₃ administration is plotted on the abscissa. The (δ¹³Cₜ) is a ¹³CO₂/¹²CO₂ concentration ratio in the expired air at each collection point in time (t) after the ¹³C—CaCO₃ administration. The (δ¹³C₀) is a ¹³CO₂/¹²CO₂ concentration ratio in the expired air before the ¹³C—CaCO₃ administration (0).

FIG. 3 shows correlations between the ¹³C—CaCO₃ dose (μmol/kg) of 4 to 200 μmol/kg and the Δ¹³C(‰) in the expired air in subjects (mammals: rats) (Experimental Example 1). FIG. 3(1) is a graph showing a correlation between the dose (μmol/kg) and the Δ¹³C(‰) in the expired air in the case where the expired air collected 5 minutes after the administration of the administration preparation was used as a test sample. FIG. 3(2) is a graph, showing a correlation between the dose (μmol/kg) and the Δ¹³C(‰) in the expired air in the case where the expired air collected 10 minutes after the administration of the administration preparation was used as a test sample. FIG. 3(3) is a graph showing a correlation between the dose (μmol/kg) and the Δ¹³C(‰) in the expired air in the case where the expired air collected 15 minutes after the administration of the administration preparation was used as a test sample.

FIG. 4 shows correlations between the ¹³C—CaCO₃ dose (μmol/kg) of 4 to 200 μmol/kg and the “area under the Δ¹³C(‰)-time curve” (AUC) determined in Experimental Example 1. The graph of the AUC for an expired air collection time of 0 to 60 minutes (AUG60) versus the ¹³C—CaCO₃ dose (4 to 200 μmol/kg) is shown on the right side, whereas the graph of the AUC for an expired air collection time of 0 to 120 minutes (AuC120) versus the ¹³C—CaCO₃ dose (4 to 200 μmol/kg) is shown on the left side.

FIG. 5(1) shows, on the left side, a correlation between the ¹³C—CaCO₃ dose (μmol/kg) of 4 to 200 μmol/kg and the Δ¹³C(‰) at an expired air collection time of 30 minutes, which was determined in Experimental Example 1; and shows, on the right side, a relation between the ¹³CaCO₃ dose (μmol/kg) of 4 to 2500 μmol/kg and the Δ¹³C(‰) at an expired air collection time of 30 minutes, which was determined in Experimental Example 1 (the ordinate is indicated by Log), FIG. 5(2) shows, on the left side, a correlation between the ¹³C—CaCO₃ dose (μmol/kg) of 4 to 200 μmol/kg and the AUC for an expired air collection time of 0 to 60 minutes (AUC60), which was determined in Experimental Example 1 (this graph is the same as the graph shown on the right side of FIG. 4). FIG. 5(2) also shows a relation between the ¹³C—CaCO₃ dose (μmol/kg) of 4 to 2500 μmol/kg and the AUC60 on the right side (the ordinate is indicated by Log).

In FIG. 6, -□- and -▪- respectively show changes in Δ¹³C(‰) (δ¹³Cₜ in the expired air at each point in time for collecting the expired air (t) after the ¹³C—CaCO₃ administration minus δ¹³C₀ in the expired air before the ¹³C—CaCO₃ administration) in the expired air measured in Group 1 (control group: normal rats) and Group 2 (model group with decreased gastric acidity) versus the time lapsed after the administration of the ¹³C—CaCO₃ suspension (point in time for collecting the expired air) (Experimental Example 2).

In FIG. 7, -□- and -▪- respectively show changes in Δ¹³C(‰) (δ¹³Cₜ in the expired air at each point in time for collecting the expired air after the ¹³C—CaCO₃ administration minus δ¹³C₀ in the expired air before the ¹³C—CaCO₃ administration) in the expired air after administering the ¹³C—CaCO₃ suspension to Group 1 (control group: normal rats) at a dose of 500 μmol/kg and after administering the ¹³C—CaCO₃ suspension to Group 2 (control group: normal rats) at a dose of 1000 μmol/kg versus time (t) after the administration. Further, and -Δ- and - ▴- respectively show changes in Δ¹³C(‰) in the expired air after administering the ¹³C—CaCO₃ suspension to Group 3 (model group with increased gastric acidity) at a dose of 500 μmol/kg, and after administering the ¹³C—CaCO₃ suspension to Group 4 (model group with increased gastric acidity) at a dose of 1000 μmol/kg versus time (t) after the administration (Experimental Example 3).

FIG. 8 shows correlations between the ¹³C—CaCO₃ dose (μmol/kg) of 4 to 1000 μmol/kg and the “area under the Δ¹³C(‰)-time curve” for an expired air collection time of 0 to 120 minutes (AUC120), which was determined in Experimental Example 3, in Group 1 (control group: normal rats, -♦-) and Group 4 (model group with increased gastric acidity, -▴-).

FIG. 9 shows changes in Δ¹³C(‰) (δ¹³Cₜ in the expired air at each collection point in time (t) after the administration of the administration solution minus δ¹³C₀ in the expired air before the administration of the administration solution) in the expired air after administrating the ¹³C—CaCO₃ suspension at 4 μmol/4 mL to rats at a rate of 4 ml per kg body weight in Experimental Example 4, and after each of the administration solutions (10, 20, 50, and 100 μmol/4 mL) of a mixture of ¹³C—CaCO₃ at each concentration of 6, 16, 46, and 96 μmol/4 mL and ¹³C—CaCO₃ at 4 μmol/4 mL was administered to the rats at a rate of 4 ml per kg body weight in Experimental Example 4.

FIG. 10 shows changes in Δ¹³C(‰) (δ¹³C value in the expired air at each collection point, in time after administration minus δ¹³C value in the expired air before administration) in the expired air after administering the ¹³C—CaCO₃ suspension at 4 μmol/4 mL to rats at a rate of 4 ml per kg body weight in Experimental Example 5 and after each of the mixtures (final concentrations: 10, 20, 50, and 100 μmol/4 mL) of ¹³C—CaCO₃ at 4 μmol/4 mL and sodium acetate was administered to the rats at a rate of 4 ml per kg body weight in Experimental Example 5.

FIG. 11 shows changes over time in Δ¹³C(‰) measured from ¹³CO₂ in the expired air after each of the ¹³C—CaCO₃ suspensions at various concentrations (400, 300, 200, 100, 50, and 20 mg/50 ml) was administered to humans (50 ml/body) in Experimental Example 6. In FIG. 11, the Δ¹³C(‰) in the expired air is plotted on the ordinate, and the expired air collection time (minutes) after the ¹³C—CaCO₃ administration is plotted on the abscissa.

FIG. 12 shows correlations between the ¹³C—CaCO₃ dose (mg/body) of 20 to 200 mg/body and the Δ¹³C(‰) in the expired air in subjects (humans) (Experimental Example 6). FIG. 12(1) shows a correlation between the dose (mg/body) and the Δ¹³C(‰) in the expired air in the case where the expired air collected 8 minutes after the administration of the administration preparation was used as a test sample. FIG. 12(2) shows a correlation between the dose (mg/body; and the Δ¹³C(‰) in the expired air in the case where the expired air collected 12 minutes after the administration of the administration preparation was used as a test sample. FIG. 12(3) shows a correlation between the dose (mg/body) and the Δ¹³C(‰) in the expired air in the case where the expired air collected 16 minutes after the administration of the administration preparation was used as a test sample.

FIG. 13 shows correlations between the ¹³C—CaCO₃ dose (mg/kg) of 20 to 200 mg/body and the “area under the Δ¹³C(‰)-time curve” (AUC), which was determined in Experimental Example 6. The graph of the AUC for an expired air collection time of 0 to 20 minutes (AUC20) versus the ¹³C—CaCO₃ dose (20 to 200 mg/body) is shown on the left side, whereas the graph of the AUC for an expired air collection rime of 0 to 60 minutes (AUC60) versus the ¹³C—CaCO₃ dose (20 to 200 mg/body) is shown on the right side.

FIG. 14 shows, on the left side, a correlation between the ¹³C—CaCO₃ dose (mg/body) of 20 to 200 mg/body and the Δ¹³C(‰) at an expired air collection time of 30 minutes, which was determined in Experimental Example 6; and shows, on the right side, a relation between the ¹³C—CaCO₃ dose (mg/body) of 20 to 400 mg/body and the Δ¹³C(‰) at an expired air collection time of 30 minutes, which was determined in Experimental Example 6 (the ordinate is indicated by Log).

FIG. 15 is a graph showing the influence of CYP2C19 and CYP3A4 inhibitor ketoconazole on the gastric acid secretion ability of proton pump inhibitor omeprazole (see Experimental Example 7).

FIG. 16 is a graph showing the influence of CYP2C19 and CYP3A4 inhibitor ketoconazole on the platelet aggregation inhibitory action of ketoconazole (see Experimental Example 8).

### DESCRIPTION OF EMBODIMENTS

### (1) Preparation Used for Measuring Gastric Acidity

The method for measuring the gastric acidity of the present invention is performed, as described later, using, as a subject, a mammal to which a ¹³C-labeled carbonate compound is orally administered.

The ¹³C-labeled carbonate compound is not limited and may be any compound that, after being orally administered to a subject, reacts with gastric acid and in some cases is degraded or metabolized in the subject's stomach, and excreted in the expired air as ¹³C-labeled carbon dioxide (¹³CO₂).

Examples of the compound that rapidly appears as ¹³C-labeled carbon dioxide in the expired air after being reacted with gastric acid in the stomach include a wide variety of ¹³C-labeled carbonate compounds that generate ¹³C-labeled carbonate ions (¹³CO₃⁻²) or ¹³C-labeled hydrogencarbonate ions (H¹³CO₃⁻¹) in a molecule when dissolved. Examples of such a ¹³C-labeled carbonate compound include carbonate compounds in a narrow sense including alkali metal carbonates such as sodium carbonate and potassium carbonate; alkaline earth metal carbonates such as calcium carbonate, magnesium carbonate, and barium carbonate; and ammonium carbonate; and hydrogencarbonate compounds including alkali metal hydrogencarbonates such as potassium hydrogencarbonate and sodium hydrogencarbonate; and ammonium hydrogencarbonate. Preferable examples include calcium carbonate, magnesium carbonate, sodium carbonate, potassium carbonate, sodium hydrogencarbonate, and potassium hydrogencarbonate. Such compounds can correctly reflect and measure gastric acid output since they are unlikely to be influenced by physiological factors such as absorption and metabolism.

Examples of ¹³C-labeled compounds other than ¹³C-labeled carbonate compounds, which appear as ¹³C-labeled carbon dioxide (¹³CO₂) in the expired air after being dissolved and then degraded or metabolized in the body, include ¹³C-labeled amino acids, proteins, organic acids, salts (e.g., alkali metal salts, such as Na) of organic acids, saccharides, lipids, and the like. These compounds generate ¹³C-labeled carbon dioxide in the expired air via the hepatic metabolism, after being digested and absorbed. Examples of the amino acids include glycine, phenylalanine, tryptophan, methionine, valine, histidine, and the like. Examples of the organic acids include acetic acid, lactic acid, pyruvic acid, butyric acid, propionic acid, octanoic acid, and their alkali metal salts. Examples of saccharides include glucose, galactose, xylose, lactose, and the like. Examples of the lipids include medium-chain triglycerides such as trioctanoin. However, these examples are not 1 imitative. Preferably, an amino acid such as glycine, an organic acid such as acetic acid or octanoic acid, or an alkali metal salt, (e.g., sodium salt or potassium salt) of such an organic acid can be used.

The method for labeling with the isotope (¹³C) is not limited, and may be a conventional one. Further, a wide variety of known or commercially available ¹³C-labeled carbonate compounds, which is labeled with such an isotope (¹³C), are usable (Sasaki, “5.1 Application of Stable Isotopes in Clinical Diagnosis”: Kagaku no Ryoiki [Journal of Japanese Chemistry] 107, “Application of Stable Isotopes in Medicine, Pharmacy, and Biology”, pp. 149-163 (1975), Nankodo: Kajiwara, RADIOISOTOPES, 41, 45-43 (1992), etc.).

The preparation to be administered to a subject (mammal) in the method of the present invention (hereinafter simply referred to as “administration preparation”) may be the aforementioned ¹³C-labeled carbonate compound per se (used singly), or may be used in the form of a composition prepared by adding, as other ingredients, for example, an excipient such as lactose, sucrose, sodium chloride, glucose, urea, starch, calcium carbonate, kaolin, crystalline cellulose, or silicic acid; a binder such as simple syrup, glucose solution, starch solution, gelatin solution, carboxymethylcellulose, shellac, methylcellulose, potassium phosphate, or polyvinyl pyrrolidone; a disintegrator such as dry starch, sodium alginate, agar powder, laminaran powder, polyoxyethylene sorbitan fatty acid esters, sodium lauryl sulfate, stearic acid monoglyceride, starch, or lactose; an absorption accelerator such as quaternary ammonium base or sodium lauryl sulfate a humectant, such as glycerin or starch; a lubricant such as purified talc, stearate, boric acid powder, or polyethylene glycol; other additives (for example, a flavor improver, taste improver, stabiliser, etc.); or the like.

The administration preparation used in the present invention may be a solid, semisolid, or liquid, and can be formulated into various forms such as powders, granules, tablets, pills, and liquids. However, it is preferable for the administration preparation used in the present invention to be dissolved promptly in the stomach. From this point of view, capsules in which a ¹³C-labeled compound is encapsulated with a capsule base are not particularly preferable. From the viewpoint of instant, solubility as well, granules, tablets, and pills are not preferably covered by a pH-dependent soluble film or a poorly soluble sugarcoating.

The amount of the administration preparation is not limited, but can be suitably selected generally from the range of 10 mg to 20 g, and preferably from the range of 10 mg to 10 g, per unit dose because such an amount of the preparation is easy to take. The amount of the ¹³C-labeled carbonate compound contained per unit dose of the administration preparation is not limited, but can be suitably selected generally from the range of 10 mg to 5 g, preferably 10 mg to 4 g, more preferably 20 mg to 2 g per body. In addition, the amount can be suitably adjusted according to the type and individual body weight of a mammalian subject. The administration is performed within 30 minutes, preferably 20 minutes, and more preferably 15 minutes before the collection of the expired air for measuring gastric acidity, generally once or twice, preferably once.

### (II) Method for Measuring Gastric Acidity

The measurement of the gastric acidity of a mammal in the present invention is non-invasively performed using, as a test sample, the expired air collected from the mammal (hereinbelow sometimes referred to as a “subject”) to which the aforementioned administration preparation (¹³C-labeled carbonate compound or preparation containing the compound) has been orally administered.

The mammals targeted by the present invention are not limited as long as the respiratory system and digestive system (particularly gastric acid secretion system of the mammals has the same function as the respiratory system and digestive system of humans. Examples thereof include humans, monkeys, dogs, cats, rabbits, guinea pigs, rats, mice, and the like. Humans are preferably used; however, when test animals are used, dogs, rabbits, guinea pigs, rats, and mice are preferable because they are easily available and easy to handle.

A method including the following steps (1) to (3) is an embodiment of the method for measuring gastric acidity.

(1) The step in which using, as a test sample, the expired air of a mammalian subject excreted at any point in time within 30 minutes after oral administration of a predetermined amount of a ¹³C-labeled carbonate compound, the behavior of ¹³CO₂ excreted in the expired air is measured.

(2) The step in which the behavior of ¹³CO₂ (hereinbelow referred to as “measured ¹³CO₂ behavior”) obtained in step (1) is compared with the behavior of the corresponding ¹³CO₂ (hereinbelow referred to as “reference ¹³CO₂ behavior”) that, has been obtained beforehand in a control mammal.

(3) The step in which the gastric acidity of the mammalian subject is determined based on the difference between the measured ¹³CO₂ behavior and the reference ¹³CO₂ behavior obtained above.

As the “¹³C-labeled carbonate compound” used in steps (1) and (2) above, those mentioned in section (I) can be used. In place of the ¹³C-labeled carbonate compound, preparations containing the ¹³C-labeled carbonate compound explained in section (I) can be used. (In the present invention, a ¹³C-labeled carbonate compound and an oral administration preparation containing the ¹³C-labeled carbonate compound are correctively referred, to as an “administration preparation.”) In this sense, the “¹³C-labeled carbonate compound” mentioned in step (1) above can also be referred to as the “administration preparation.”

The respective steps in the method for measuring gastric acidity are explained below.

### (1) Step of Measuring the Behavior of ¹³CO₂ Excreted in the Expired Air

Step (1) is a step of collecting expired air from a subject (mammal) to which a predetermined amount of an administration preparation (a ¹³C-labeled carbonate compound or a preparation containing the compound) has been orally administered beforehand, and measuring the behavior of ¹³CO₂ contained in the collected expired air.

The method and the timing of orally administering the administration preparation to the subject are not limited. To measure basic gastric acidity (fasting gastric acidity), the preparation is preferably administered on an empty stomach to avoid the influence of foods. More preferably, it is desirable that the preparation be orally administered for 4 hours, even more preferably 10 hours after the start of fasting. However, to measure gastric acidity under general physiological conditions while reducing variations in the measured values between individuals, it is preferable to measure gastric acidity after stimulation of gastric, acid secretion. In this case, it is preferable to administer a test beverage (e.g., water, beverage containing caffeine, alcoholic beverage, and consommé soup; and solid or liquid foods such as Calorie Mate (registered trademark)) or inject a gastric acid secretion stimulant (e.g., histamine hydrochloride, betazole hydrochloride, gastrin, insulin, etc.) to stimulate gastric acid secretion, and then orally administer a preparation at least one hour after the administration or injection,

When the administration preparation orally administered to the subject enters into the stomach, it dissolves with gastric juice to release a ¹³C-labeled carbonate compound, and the ¹³C-labeled carbonate compound is reacted with gastric acid to form ¹³C-labeled carbon dioxide (¹³CO₂). The ¹³C-labeled carbon dioxide is gradually excreted in the expired air.

A reaction formula to form ¹³C-labeled carbon dioxide (¹³CO₂) is shown below using ¹³C-labeled calcium carbonate (Ca¹³CO₃) as the ¹³C-labeled carbonate compound.

Ca¹³CO₃+2HCl→CaCl₂+H₂O+¹³CO₂↑

The “behavior of ¹³CO₂” measured in the present invention is, for example, the following (a) to (d):

(a) The amount of ¹³CO₂ excreted in the expired air at any point in time within 30 minutes after the oral administration of the administration preparation (including a preparation containing a ¹³C-labeled carbonate compound alone, the same as below).

(b) The ratio of ¹³CO₂ amount to ¹²CO₂ amount excreted in the expired air at any point in time within 30 minutes after the oral administration of the predetermined amount of the administration preparation ((¹³CO₂/¹²CO₂ concentration ratio; δ¹³C).

(c) The difference [Δ¹³C(‰)=δ¹³Cₜ−δ¹³C₀] (hereinbelow referred to as “Δ¹³C(‰)ₜ” or “Δ¹³C(‰)”) between the “ratio of ¹³CO₂ amount to ¹²CO₂ amount” (hereinbelow referred to as “δ¹³Cₜ”) included in the expired air collected at any point in time for collecting the expired air (t) within 30 minutes after the oral administration of a predetermined amount of the administration preparation, and the “ratio of ¹³CO₂ amount to ¹²CO₂ amount” (hereinbelow referred to as “δ¹³C₀”) included in the expired air before the oral administration of the administration preparation.

(d) AUC “area under the Δ¹³C(‰)-time curve” calculated by making a graph by plotting the time from the administration of the predetermined amount of the administration preparation to the collection of the expired air on the abscissa and the Δ¹³C(‰) on the ordinate.

The behavior of ¹³CO₂ is preferably Δ¹³C(‰) and the “area under the Δ¹³C(‰) time-curve” (AUC), and more preferably Δ¹³C(‰).

Specifically, the behavior of such ¹³CO₂ can be measured as follows.

After the predetermined amount of the administration preparation is orally administered to a subject, the expired air is collected according to a conventional ¹³C expiration test method (Kajiwara, RADIOISOTOPES, 41, 45-48 (1992); Kajiwara et al., RADIOISOTOPES, 41, 331-334 (1992), etc.). The amount of ¹³CO₂ excreted in the expired air is measured as the “¹³CO₂/¹²CO₂ concentration ratio (δ¹³C)” at each point in time for collecting the expired air (t) (“δ¹³Cₜ”: the ratio of ¹³CO₂ amount to ¹²CO₂ amount (carbon dioxide) excreted in the expired air, t represents an expired air collection time (a time lapsed after the administration of the administration preparation)).

Subsequently, based on the difference between “δ¹³Cₜ” and the reference “¹³CO₂/¹²CO₂ concentration ratio (δ¹³C)” (hereinbelow sometimes referred to as “δ¹³C₀”), which has been measured beforehand prior to the administration of the administration preparation, “Δ¹³C(‰)” [Δ¹³C(‰)=δ¹³Cₜ−δ¹³C₀] is calculated. The behavior of ¹³CO₂ excreted in the expired air at any point in time (f) within 30 minutes after the administration of the preparation can be obtained according to the above formula [Δ¹³C(‰)=δ¹³Cₜ−δ¹³C₀] (“t” means an expired air collection time within 30 minutes from the administration of the administration preparation).

The behavior of ¹³CO₂ excreted in the expired air over time can be obtained by tracing the change of Δ¹³C(‰) over time. Specifically, the behavior of ¹³CO₂ can be obtained by making a graph by plotting the expired air collection time (min); in other words, the lapse of time (min) after the administration of the preparation, on the abscissa and the Δ¹³C(‰) on the ordinate.

The labeled substance (¹³CO₂) contained in the collected expired air can be measured and analyzed by a conventional analysis technique, such as liquid scintillation counting, mass spectroscopy, infrared spectroscopic analysis, emission spectrochemical analysis, or magnetic resonance spectral analysis. From the viewpoint of measurement accuracy, infrared spectroscopic analysis and mass spectrometry are preferable.

Moreover, the “area under the Δ¹³C(‰)-time curve” (AUC) can be obtained by calculating the area tinder the curve based on the graph in which the lapse of time (min) after the administration of the administration preparation (expired air collection time; t) is plotted on the abscissa and the Δ¹³C(‰) is plotted on the ordinate,

The timing of expired air collection is generally at least 10 seconds and preferably at least one minute after the oral administration of the administration preparation. The time until the expired air is collected after the administration of the administration preparation is preferably within 30 minutes, more preferably 20 minutes, and even more preferably 15 minutes. For example, the expired air can be collected once or twice in the range of 10 seconds to 30 minutes, preferably 1 minute to 20 minutes, and more preferably 1 minute to 15 minutes after the oral administration of the administration preparation, and used as a test sample. The expired air is preferably collected once.

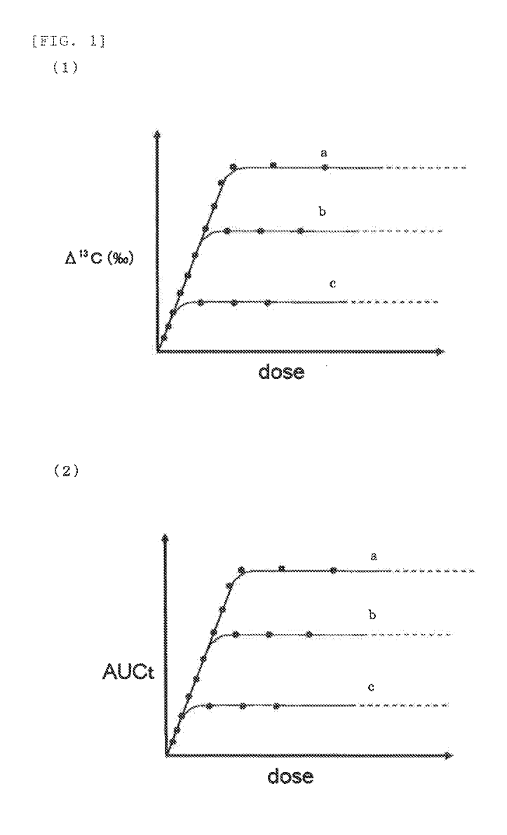

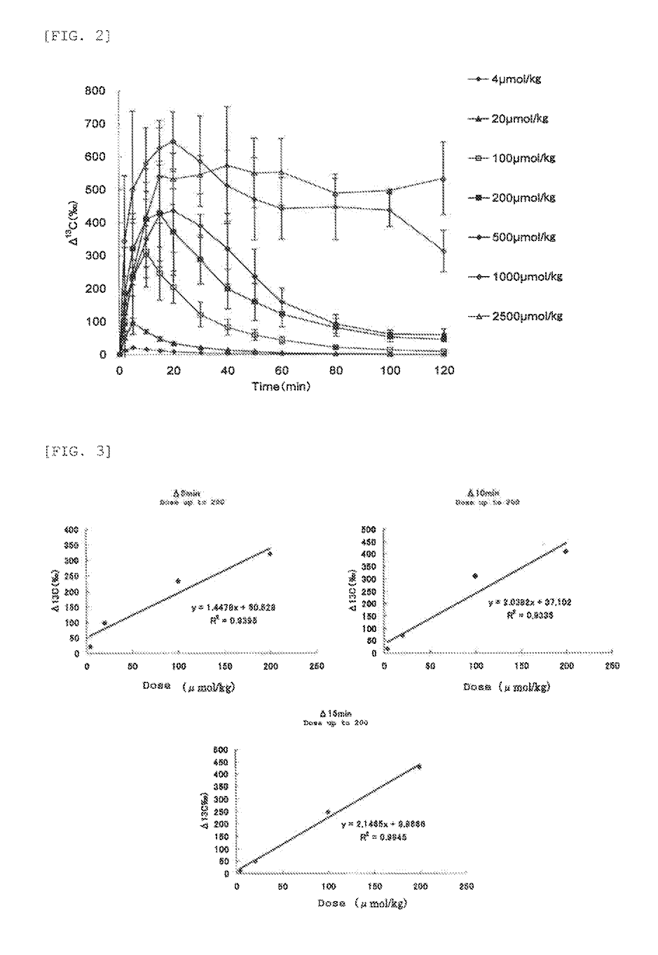

FIG. 1 (1) and (2) schematically illustrate the results of the thus obtained Δ¹³C(‰) versus the dose of the administration preparation and the “area under the Δ¹³C(‰)-time curve” (AUC) versus the dose of the administration preparation, respectively. In FIG. 1, a shows the correlation between the dose and the Δ¹³C(‰) or AUC of a mammal having higher gastric acidity than normal; b shows the correlation between the dose and the Δ¹³C(‰) or AUC of a mammal having normal gastric acidity; and c shows a correlation between the dose and the Δ¹³C(‰) or AUC of a mammal having lower gastric acidity than normal.

As shown in the figure, regardless of the degree of gastric acidity, in all subjects having normal gastric acidity, subjects (mammals) having gastric acidity higher than the normal acidity (including, for example, patients with “hyperacidity”), and subjects (mammals) having gastric acidity lower than the normal acidity (including, for example, patients with “hypoacidity or anacidity,” the correlation passing through the origin is seen between the dose of the administration preparation and Δ¹³C(‰), or between the dose and AUC up to a certain amount; however, the value becomes constant when the dose exceeds a certain amount. Such a certain amount is referred to as “Δ¹³C(‰) plateau value” or “AUC plateau value,” and the dose at which the Δ¹³C(‰) or AUC reaches the plateau value is referred to as “reference dose.”

The “Δ¹³C(‰) plateau value” and the “reference dose” vary depending on the subject's (mammalian) gastric acidity. Specifically, as shown in FIG. 1 (1), the “Δ¹³C(‰) plateau value” and the “reference dose” depend on the subject's (mammalian) gastric acidity. When the “Δ¹³C(‰) plateau value” and the “reference dose” of normal gastric acidity are regarded as standard values (hereinbelow referred to as “normal Δ¹³C(‰) plateau value” and “normal reference dose” for convenience in the present specification), a subject (subject with high gastric acidity) who has higher gastric acidity than normal, such as a patient with hyperacidity has a higher Δ¹³C(‰) plateau value than the normal Δ¹³C(‰) plateau value, and a higher reference dose than the normal reference dose. A subject (subject with low gastric acidity) who has lower gastric acidity than normal, such as a patient with hypoacidity or anacidity has a lower Δ¹³C(‰) plateau value than the normal Δ¹³C(‰) plateau value, and a lower reference dose than the normal reference dose.

The “Δ¹³C(‰) plateau value” and the “reference dose” of a subject (mammal) having higher gastric acidity than normal are referred to as “high-acidity Δ¹³C(‰) plateau value” and “high-acidity reference dose” for convenience. The “Δ¹³C(‰) plateau value” and the “reference dose” of a subject (mammal) having lower gastric acidity than normal are referred to as “low-acidity Δ¹³C(‰) plateau value” and “low-acidity reference dose” for convenience.

The “Δ¹³C(‰) plateau value” (normal Δ¹³C(‰) plateau value, high-acidity Δ¹³C(‰) plateau value, and low-acidity Δ¹³C(‰) plateau value) and the “reference dose” (normal reference dose, high-acidity reference dose, end low-acidity reference dose) can be obtained by performing the following steps (a) to (c) on a control mammal beforehand, and making a graph (hereinbelow referred to as a “dose-Δ¹³C(‰)” plot) in which the administration preparation dose is plotted on the abscissa, and the Δ¹³C(‰) is plotted on the ordinate.

(a) The step in which an administration preparation including a preparation containing a ¹³C-labeled carbonate compound alone) is orally administered to a control mammal at a dose ranging from 0 to 2500 (dose), and the amount of ¹³CO₂ excreted in the expired air of the mammal is measured for each dose.

(b) The step in which Δ¹³C(‰)ₜ is obtained by calculating a difference (Δ¹³C(‰)=δ¹³Cₜ−δ¹³C₀) between the ratio of ¹³CO₂ amount to ¹²CO₂ amount (¹³CO₂/¹²CO₂ concentration ratio) (“δ¹³Cₜ”) in the expired air at a point in time for collecting the expired air (t) and the ratio of ¹³CO₂ amount to ¹²CO₂ amount (¹³CO₂/¹²CO₂ concentration ratio) (“δ¹³C₀”) in the expired air before the administration, based on the amount of ¹³CO₂ obtained above.

(c) The step in which Δ¹³C(‰)ₜ obtained in step (b) above is plotted against the dose of the administration preparation to form a calibration curve.

The control mammal used herein is preferably the same kind of animal as the mammalian subject. For example, when the measurement subject (mammal) is a human, the control subject is preferably a human (mammal); and when the measurement subject (mammal) is a rat, the control subject is preferably a rat (mammal). Although there is no limitation, the sex, age, weight, and the like of the control subject preferably correspond to those of the mammalian subject. In the above, the unit of dose (0 to 2500) is, for example, “μmol/kg” or “mg/body.” As shown in the examples, when test animals such as rats are used, the unit. “μmol/kg” can be used; and, for humans, the unit “mg/body” is preferably used.

The normal Δ¹³C(‰) plateau value and the normal reference dose can be calculated using a mammal (normal mammal) having normal gastric acidity as a control mammal. In general, common mammals have normal gastric acidity; however, for example, it may be better to confirm beforehand whether the control mammal has normal gastric acidity using the other measurement methods described, for example, in “Stomach, intestinal, pancreatic function examinations” (Outline of Clinical Examination, 33^{rd} edition, Kanehara & Co. Ltd.) and Patent Literature 1. The gastric acidity used in the present invention corresponds to “[the hydrochloric acid concentration in the stomach (mEq/L)]×[gastric juice volume (L)]”

The high-acidity Δ¹³C(‰) plateau value and the high-acidity reference dose can be calculated using a mammal having higher gastric acidity than normal as a control mammal. Mammals having high gastric acidity can be distinguished by the other gastric acidity measurement methods (see Patent Literature 1 and the references mentioned above). Alternatively, mammals having high gastric acidity can be prepared using an artificial measure, i.e., administering to the normal mammal a drug that promotes and increases gastric acid secretion, as shown, in Experimental Example 3 mentioned later, to increase gastric acidity. The thus-obtained mammals can be used.

The low-acidity Δ¹³C(‰) plateau value and the low-acidity reference dose can be calculated using a mammal having lower gastric acidity than normal as a control mammal. Mammals having low gastric acidity can be distinguished by the other gastric acidity measurement methods (see Patent Literature 1 and the references mentioned above). Alternatively, mammals having low gastric acidity can be prepared using an artificial measure, i.e., administering to the normal mammal a drug that inhibits gastric acid secretion, as shown in Experimental Example 2 mentioned later, to reduce gastric acidity. The thus-obtained mammals can be used.

In step (1), the dose of the administration preparation (a ¹³C-labeled carbonate compound or a preparation containing the compound) that has been administered beforehand to a subject (mammal) in step (1) can be determined based on the “dose-Δ¹³C(‰) plot” prepared for the control mammal beforehand, or the “reference dose” (normal reference dose, high-acidity reference dose, and low-acidity reference dose) obtained from, the plot. In the present invention, this is called a “predetermined dose.” The predetermined dose used in step (1) is generally lower than the high-acidity reference dose, preferably in the range of the low-acidity reference dose to the high-acidity reference dose, and more preferably the normal reference dose or approximately the normal reference dose (normal reference dose ±100 μmol/kg or ±100 mg/body). The normal reference dose is not limited, and may be about 200 μmol/kg or 200 mg/body,

Thus, the relation between the “Δ¹³C(‰) plateau value” and the “reference dose” is explained. As shown in FIG. 1, a similar relation can be seen between the “AUC plateau value” and the “reference dose”; therefore, the AUC plateau value can be used in place of the Δ¹³C(‰) plateau value.

### (2) Step of Comparing the Measured ¹³CO₂ Behavior and the Reference ¹³CO₂ Behavior

### (3) Step of Determining the Gastric Acidity of the Mammalian Subject

Step (2; is a step of comparing the behavior of ¹³CO₂ (measured ¹³CO₂ behavior) obtained in step (1) with the behavior of the corresponding ¹³CO₂ (reference ¹³CO₂ behavior; that has been obtained beforehand in a control mammal.

Step (3) is a step of determining the gastric acidity of a mammalian subject based on the difference between the measured ¹³CO₂ behavior and the reference ¹³CO₂ behavior obtained in step (2).

The “behavior of ¹³CO₂” may be the following (a) to (d), and the behavior of ¹³CO₂ measured in the same kind of animal as the mammalian subject whose behavior of ¹³CO₂ (measured ¹³CO₂ behavior) is measured, is used as the reference ¹³CO₂ behavior.

(a) The amount of ¹³CO₂ excreted in the expired air at any point, in time within 30 minutes after the oral administration of the predetermined amount of the administration preparation (including a preparation containing a ¹³C-labeled carbonate compound alone, the same as below).

(b) The ratio of ¹³CO₂ amount to ¹²CO₂ amount excreted in the expired air at any point in time within 30 minutes after the oral administration of the predetermined amount of the administration preparation ((¹³CO₂/¹²CO₂ concentration ratio: δ¹³C).

(c) Difference [Δ¹³C(‰)=δ¹³Cₜ−δ¹³C₀] between the “ratio of ¹³CO₂ amount to ¹²CO₂ amount” (δ¹³C₀) included in the expired air collected at any point in time (t) for collecting the expired air within 30 minutes after the oral administration of the predetermined amount of the administration preparation and the “ratio of ¹³CO₂ amount to ¹²CO₂ amount” (δ¹³C₀) included in the expired air before the oral administration of the administration preparation.

(d) AUC “area under the Δ¹³C(‰)-time curve” calculated by making a graph by plotting the time from the administration of the predetermined amount of the administration preparation to the collection of the expired air on the abscissa and the Δ¹³C(‰) on the ordinate.

The behavior of ¹³CO₂ is preferably Δ¹³C(‰) and AUC, and more preferably Δ¹³C(‰).

For example, when the Δ¹³C(‰) is used as the “behavior of ¹³CO₂,” the gastric acidity of the mammalian subject can be quantified by the following method.

(i) As a predetermined dose, the normal reference dose of the administration preparation is administered to a mammalian subject.

(ii) The expired air is collected at any point in time within 30 minutes after the administration, and the Δ¹³C(‰) (this is called “measured Δ¹³C(‰)”) is obtained based on the amount of ¹³CO₂ excreted in the expired air.

(iii) From the “dose-Δ¹³C(‰)” plot (calibration curve indicating the relation between the administration preparation dose and Δ¹³C(‰), which has been produced beforehand for the control mammal having normal gastric acidity, Δ¹³C(‰) (referred to as “reference Δ¹³C(‰)”) corresponding to the normal reference dose is obtained, and compared with the measured Δ¹³C(‰).

(iv) When the measured Δ¹³C(‰) is lower than the reference Δ¹³C(‰), the gastric acidity of the mammalian subject can be determined to be lower than the normal value.

(v) When the measured Δ¹³C(‰) is the same as the reference Δ¹³C(‰), the gastric acidity of the mammalian subject can be determined to be the same as or higher than the normal value.

For example, with reference to the left figure of FIG. 12 (Experimental Example 6), which is the “dose-Δ¹³C(‰)” plot in which Ca¹³CO₃ at a dose up to 200 mg/body is orally administered to a human, when the Δ¹³C(‰) is a value (140) that intersects with the “dose-Δ¹³C(‰)” plot at 8 minutes after the oral administration of 150 mg/body of the administration preparation (Ca¹³CO₃), the gastric acidity is determined to be normal or higher than normal; and when the Δ¹³C(‰) is lower than that value (140), for example, 100, the gastric acidity is determined to be lower than normal.

In this case, the gastric acidity of the mammalian subject can be calculated as follows.

(a) Obtain the dose (mg/body or μmol/kg) (hereinbelow, referred to as “calcium carbonate equivalent amount”) at which the “dose-Δ¹³C(‰)” plot intersects with the measured Δ¹³C(‰) value.
(b) Calculate the gastric acidity (M or Eq) using the calcium carbonate equivalent amount obtained in step (a) (mg/body or μmol/kg) according to the following formula.

Gastric acidify (mM[mmol])=[calcium carbonate equivalent amount (mg/body)×2]/molecular weight of CaCO₃; or

Gastric acidity (μ Eq)=calcium carbonate equivalent amount (μmol/kg)×weight of mammalian subject×2

Based on the above, in the “dose-Δ¹³C(‰)” plot in the upper-left figure of FIG. 12, the calcium carbonate equivalent amount (mg/body) of the subject whose measured Δ¹³C(‰) is 100 is determined to be 100 mg/body; therefore, the gastric acidity of the subject is 2 mM according to the following formula.

Gastric acidity (mM [mmol])=[100 mg/body×2]/100 (molecular weight of CaCO₃).

As another method, when the “area under the Δ¹³C(‰)-time curve” (AUC) is used as the “behavior of ¹³CO₂,” the gastric acidity of the mammalian subject can be quantified by the following method.

(i) As a predetermined dose, the normal reference dose of the administration preparation is administered to a mammalian subject.

(ii) After the administration, the expired air is collected over time, and the Δ¹³C(‰) is obtained based on the amount of ¹³CO₂ excreted in the expired air at a predetermined period. The “area under the Δ¹³C(‰)-time curve” (AUC) (referred to as “measured AUG”) is obtained by making a graph by plotting the time from the administration to the collection of the expired air on the abscissa and the Δ¹³C(‰) on the ordinate.

(iii) From the “dose-AUC” plot (calibration curve indicating the relation between the dose of the administration preparation and the area under the Δ¹³C(‰)-time curve (AUC)), which has been produced beforehand for the control mammal, AUC (referred to as “reference AUG”) corresponding to the normal reference dose is obtained, and compared with the measured AUC.

(iv) When the measured AUC is lower than the reference AUC, the gastric acidity of the mammalian subject can be determined to be lower than the normal value.

(v) When the measured AUC is the same as the reference AUC, the gastric acidity of the mammalian subject; can be determined to be the same as or higher than the normal value.

In this case, similar to the case where the Δ¹³C(‰) is used as the “behavior of ¹³CO₂”, the gastric acidity of the mammalian subject can be quantified as follows.

(a) Obtain the dose (mg/body or μmol/kg) (hereinbelow, referred to as “calcium carbonate equivalent amount”) at which the “dose-AUC” plot intersects with the measured AUC value.

(b) Calculate the gastric acidity (M or Eq) using the calcium carbonate equivalent amount obtained in step (a) according to the following formula,

Gastric acidity (mM[mmol])=[calcium carbonate equivalent amount (mg/body)×2]/molecular weight of CaCO₃; or

Gastric acidity (μ Eq)=calcium carbonate equivalent amount (μmol/kg)×weight of mammalian subject×2

The method for measuring the gastric acidity of the present invention explained above car; be used as a method for diagnosing or evaluating the presence or absence of a decrease or increase of the basic gastric acid secretion of mammals.

The method for measuring the gastric acidity of the present invention, particularly, the method using Δ¹³C(‰) as the “¹³CO₂ behavior” is useful in that the method can non-invasively evaluate and diagnose the gastric acidity in a simple manner with few expired-air collections (preferably one time), and without restraining a subject over a long period of time. By using the measurement method of the gastric acidity of the present invention, gastric acidity tendency (gastric hyperacidity, normal, hypoacidity, anacidity, etc.) can be measured, and consequently, diseases involving gastric acid secretion can be diagnosed. Further, the drug effect or treatment effect of the gastric acid reducer can be evaluated. Specifically, the evaluation can be performed by measuring the gastric acidity before and after the administration of the gastric acid reducer to a subject using the administration preparation of the present invention, and comparing the results. By this method, the drug effect of the administration drug (gastric acid reducer), and the treatment effect of each drug on the subject can be evaluated. Consequently, the method can be used as a means for selecting a drug (gastric acid reducer) suitable for each subject.

In addition, as a gastric acid reducer, drugs having an effect of increasing gastric pH, including gastric acid secretion inhibitors such as proton pump inhibitors (PPI) and H₂ blockers, and drugs having an effect of neutralizing gastric acid such as antacids can be used. Therefore, the present invention can be used as a method for detecting a gastric pH change by the drugs to evaluate the effect of the drugs on a subject,

To perform the method of the present invention, when the subject's gastric acidity is predicted to be high beforehand, the gastric acidity of the subject can be accurately or economically evaluated by using the high-acidity reference dose as a predetermined dose. When the effect of the gastric acid reducer on a subject having high gastric acidity is evaluated, since a decrease in the gastric acidity due to the administration of such a drug is anticipated, the normal reference dose is used as a predetermined dose, and the effect of the drug can thereby be accurately or economically evaluated. When the subject's gastric acidity is predicted to be low beforehand, the gastric acidity of the subject car; be economically evaluated by using the low acid reference dose as a predetermined dose.

Of the gastric acid reducers, proton pump inhibitors such as omeprazole and lansoprazole are drugs metabolized by hepatic metabolism enzymes such as CYP2C19 and CYP3A4. Therefore, the effects of these drugs depend on the enzyme activity (metabolic activity) of CYP2C19 and CYP3A4 in a subject; however, since these enzymes have a plural of genetic polymorphism, and the enzyme activity (metabolic capacity) is different depending on the gene tit: polymorphism, the effect of omeprazole and that of lansoprazole are different. By using the gastric acidity measurement method of the present invention, the enzyme activity (metabolic activity) of CYP2C19 and CYP3A4 in a subject can be measured and evaluated based on the gastric acidity after the administration of omeprazole or lansoprazole. Even when drugs other than omeprazole and lansoprazole are used, by evaluating the gastric acidity after the administration of omeprazole or lansoprazole, the susceptibility of a subject to drugs whose activity is eliminated by being metabolized by CYP2C19 and CYP3A4, or drugs that exhibit activity by being metabolized by such enzymes (e.g., Plavix) can also be evaluated.

### (III) Method for Measuring the Effect of a Gastric Acid Reducer

As indicated above, by using the gastric acidity measurement method of the present invention, the effect of the gastric acid reducer on a mammalian subject, can be measured. Specifically, the effect-measuring method can be performed by the following steps (1) to (4).

(1) The step in which using, as a test sample, the expired air of a mammalian subject excreted at any point in time within 30 minutes after oral administration of a predetermined amount of a ¹³C-labeled carbonate compound, the oral administration being performed after administration of a gastric acid reducer, the behavior of ¹³CO₂ excreted in the expired air is measured.

(2) The step in which the behavior of ¹³CO₂ (measured ¹³CO₂ behavior) obtained in step (1) is compared with the behavior of corresponding ¹³CO₂ (reference ¹³CO₂ behavior) measured in a mammal (control mammal) to which a predetermined amount of a ¹³C-labeled carbonate compound has been orally administered beforehand without administering the gastric acid reducer.

(3) The step in which the gastric acidity of the mammalian subject is determined based on the difference between the reference ¹³CO₂ behavior and the measured ¹³CO₂ behavior obtained above.

(4) The step in which the effect of the gastric acid reducer on the mammalian subject is determined using the gastric acidity of the mammalian subject obtained, above as an index.

The gastric acid reducer includes a proton pump inhibitor and an H₂ blocker each having an effect of inhibiting gastric acid secretion, and an antacid having an effect of neutralizing gastric acid.

A proton pump inhibitor is a drug that acts on proton pump of gastric parietal cells, and inhibits gastric acid secretion. Examples of the drugs include omeprazole, lansoprazole, pantoprazole, rabeprazole, esomeprazole, and the like. An H₂ blocker is a drug that inhibits gastric acid secretion by competitively antagonizing a histamine H₂ receptor, which is present in gastric parietal cells and enhances gastric acid secretion. Examples thereof include ranitidine, cimetidine, famotidine, nizatidine, lafutidine, roxatidine acetate hydrochloride, and the like. Further, an antacid is a drug that has an effect of neutralizing overproduced gastric: acid to adjust gastric pH. Examples thereof include magnesium hydroxide, anhydrous dibasic calcium phosphate, precipitated calcium carbonate, sodium hydrogencarbonate, magnesium oxide, and the like.

Step (1), in which the behavior of ¹³CO₂ excreted in the expired air is measured, can be performed on a mammalian subject to which a ¹³C-labeled carbonate compound is orally administered after the administration of a gastric acid reducer whose effect is an interest of evaluation. The mammalian subject is preferably a human, as in the method for measuring gastric acidity. Test animals such as monkeys, dogs, cats, rabbits, guinea pigs, rats, mice, etc., can also be used.

The interval between the administration of the gastric acid reducer and the administration of the ¹³C-labeled carbonate compound to a mammalian subject is not limited, but is generally in the range of 1 minute to 12 hours, preferably 2 to 480 minutes, and more preferably 5 to 240 minutes.

Except for the use of the mammalian subject to which a gastric acid reducer is administered before the administration of a ¹³C-labeled carbonate compound, step (1) is performed in the same manner as in the method explained in Item (1) of the section “(II) Method for measuring gastric acidity” above. Similarly, as the ¹³C-labeled carbonate compound, the administration preparation explained in the section “(I) Preparation used for measuring gastric acidity” can be used.

Step (2), in which the measured ¹³CO₂ behavior and the reference ¹³CO₂ behavior are compared, is a step of comparing the ¹³CO₂ behavior (measured ¹³CO₂ behavior) obtained in step (1) and the corresponding ¹³CO₂ behavior (reference ¹³CO₂ behavior) that has been obtained beforehand in a control mammal. The control mammal used herein is a mammal to which a predetermined amount of a ¹³C-labeled carbonate compound is orally administered as in the mammalian subject, without administering a gastric acid reducer, and is generally the same kind of mammalian subject, as explained in Item (II) above. There is no particular limitation, but the control mammal preferably has the same sex, and almost the same age and weight as the mammalian subject.

Except for the use of the ¹³CO₂ behavior obtained in the above-mentioned control mammal as the referent ¹³CO₂ behavior, step (2) can use the same method explained in Items (1) and (2) of the section “(II) Method for measuring gastric acidity.”

Step (3), which determines the gastric acidity, can be performed based on a difference between the reference 1 behavior and the measured ¹³CO₂ behavior obtained in step (2), and can use the same method explained in Items (2) and (3) of the section “(II) Method for measuring gastric acidity.”

Step (4), which determines the effect of the gastric acid reducer, is carried out using the gastric acidity of the mammalian subject obtained in step (3) as an index.

Specifically, in step (4), when the gastric acidity of the mammalian subject measured in step (3) is the same as or higher than the gastric acidity of the control mammal, the administered gastric acid, reducer can be determined as having no effect on the mammalian subject. When the gastric acidity of the mammalian subject, measured in step (3) is lower than the gastric acidity of the control mammal, the administered gastric acid reducer can be determined as having an effect on a mammalian subject.

(IV) Method for Evaluating the Enzyme Activity (Metabolic Capacity) of CYP2C19 Alone or Both CYP2C19 and CYP3A4 in a Mammalian Subject, Effect of a Drug Metabolized by CYP2C19 Alone or Both CYP2C19 and CYP3A4 on a Mammalian Subject, or/and Susceptibility of a Mammalian Subject to the Drug

As indicated above, using the gastric acidity measurement method of the present, invention, the enzyme activity (metabolic capacity) of CYP2C19 alone or both CYP2C19 and CYP3A4 in the mammalian subject, effect metabolized by CYP2C19 alone or both CYP2C19 and CYP3A4 on the mammalian subject, or/and susceptibility of the mammalian subject to the drug can be evaluated.

Specifically, the evaluation method can be carried out by performing the following steps (1) to (4).

(1) The step in which using, as a test sample, the expired air of a mammalian subject excreted at any point in time within 30 minutes after oral administration of a predetermined amount of a ¹³C-labeled carbonate compound, the oral administration being performed after administration of omeprazole or lansoprazole, the behavior of ¹³CO₂ excreted in the expired air is measured.

(2) The step in which the behavior of ¹³CO₂ (measured ¹³CO₂ behavior) obtained in step (I) is measured with the behavior of corresponding is ¹³CO₂ (reference ¹³CO₂ behavior) measured in a mammal (control mammal) to which a predetermined amount of a ¹³C-labeled carbonate compound has been orally administered beforehand without administering the omeprazole or lansoprazole.

(3) The step in which the gastric acidity of the mammalian subject is determined based on the difference between the reference ¹³CO₂ behavior and the measured ¹³CO₂ behavior obtained above.

(4) The step in which the enzyme activity (metabolic capacity) of CYP2C19 alone or both CYP2C19 and CYP3A4 in the mammalian subject, effect metabolized by CYP2C19 alone or both CYP2C19 and CYP3A4 on the mammalian subject, or/and susceptibility of the mammalian subject to the drug are determined using the gastric acidity of the mammalian subject obtained above as an index.

Here, omeprazole and lansoprazole are both drugs that are metabolized in the body by the effects of CYP2C19 and CYP3A4, which are hepatic metabolism enzymes.

Step (1), in which the behavior of ¹³CO₂ excreted in the expired air is measured, can be performed on a mammalian subject to which a ¹³C-labeled carbonate compound is orally administered after the administration of omeprazole or lansoprazole. The mammalian subject is preferably a human, as in the method for measuring gastric acidity. Test animals such as monkeys, dogs, cats, rabbits, guinea pigs, rats, mice, etc., can also be used.

The interval between the administration of omeprazole or lansoprazole and the administration of the ¹³C-labeled carbonate compound to the mammalian subject is not limited, but it is generally in the range of 1 minute to 12 hours, preferably 2 to 480 minutes, and more preferably 5 to 240 minutes.

Except for the use of the mammalian subject to which omeprazole or lansoprazole is administered before the administration of a ¹³C-labeled carbonate compound, step (15 is performed in the same manner as in the method explained in Item (1) of the section “(II) Method for measuring gastric acidity” above. Similarly, as the C-labeled carbonate compound, the administration preparation explained in the section “(I) Preparation used for measuring gastric acidity” can be used.

Step (2) in which the measured ¹³CO₂ behavior and the reference ¹³CO₂ behavior are compared is a step of comparing the ¹³CO₂ behavior (measured ¹³CO₂ behavior) obtained in step (1) and the corresponding ¹³CO₂ behavior (reference ¹³CO₂ behavior) that has been obtained, beforehand in a control mammal. The control mammal used herein is a mammal to which a predetermined amount of a ¹³C-labeled carbonate compound is orally administered as in the mammalian subject, without administering omeprazole or lansoprazole; and, as explained in Item (II) above, the control mammal is generally the same kind of the mammalian subject. There is no particular limitation, but the control mammal, preferably has the same sex, and almost the same age and weight as the mammalian subject.

Except for the use of the ¹³CO₂ behavior obtained in the above-mentioned control mammal as the reference ¹³CO₂ behavior, step (2) can use the same method as explained in Items (1) and (2) of the section “(II) Method for measuring gastric acidity.”

Step (3) that determines the gastric acidity can be performed based on a difference between the reference ¹³CO₂ behavior obtained in step (2) and the measured ¹³CO₂ behavior, and can use the same method as explained in Items (2) and (3) of the section “(II) Method for measuring gastric acidity.”

Using the gastric acidity of the mammalian subject obtained in step (3) as an index, step (4), i.e., (a) determining the enzyme activity (metabolic capacity) of CYP2C19 alone or both CYP2C19 and CYP3A4 in the mammalian subject, (b) determining the effect of the drug metabolised by CYP2C19 alone or both CYP2C19 and CYP3A4, or/and (c) determining the susceptibility of the mammalian subject, to the drug can be performed.

Step (4) may be a step of determining any one of or at least two items of above (a) to (c) relating to the mammalian subject, using the gastric acidity of the mammalian subject as an index. Preferably, it is the step of determining the enzyme activity (metabolic capacity) of CYP2C19 alone or both CYP2C19 and CYP3A4 in the mammalian subject (step (a)) , the step of determining the effect of a drug metabolized by CYP2C19 alone or both CYP2C19 and CYP3A4 (step (b)), and the step of performing step (b) after step (a).

In the case where step (4) includes step (a) of determining the enzyme activity (metabolic capacity) of CYP2C19 alone or both CYP2C19 and CYP3A4 in a subject mammal, when the gastric acidity of the mammalian subject measured in step (3) is the same as or higher than that of the control mammal, step (4) can determine that the enzyme activity (metabolic capacity) of CYP2C19 alone or both CYP2C19 and CYP3A4 in the mammalian subject is normal or higher than normal. Alternatively, when the gastric acidity of the mammalian subject measured in step (3) is lower than that of the control mammal, step (4) can determine that the enzyme activity (metabolic capacity) of CYP2C19 alone or both CYP2C19 and CYP3A4 in the mammalian subject is lower than normal.

In the case where step (4) includes step (b) of determining the effect of the drug metabolized by CYP2C19 alone or both CYP2C19 and CYP3A4, the effect of the drug is different when the drug loses or reduces its activity when metabolized by CYP2C19 alone or both CYP2C19 and CYP3A4, and when the drug expresses or increases its activity when metabolized by CYP2C19 alone or both CYP2C19 and CYP3A4.

In the case where the drug that loses or reduces its activity when metabolized by CYP2C19 alone or both CYP2C19 and CYP3A4 is used, as a drug, when the gastric acidity of the mammalian subject measured in step (3) is the same as or higher than that of the control mammal, step (4) can determine that the effect of the drug on the subject is low; and when the gastric acidity of the mammalian subject measured in step (3) is lower than that of the control mammal, step (4) can determine that the effect of the drug on the subject is high.

On the other hand, in the case where the drug that expresses or increases its activity when metabolized by CYP2C19 alone or both CYP2C19 and CYP3A4 is used as a drug, when the gastric acidity of the mammalian subject measured in step (3) is the same as or higher than, that of the control mammal, step (4) can determine that the effect of the drug on the mammalian subject is high; and when the gastric acidity of the mammalian subject measured in step (3) is lower than that, of the control mammal, step (4) can determine that, the effect of the drug on the subject is low.

In the case where step (4) includes step (c) of determining the susceptibility of the mammalian subject to the drug metabolized by CYP2C19 alone or both CYP2C19 and CYP3A4, the susceptibility is different when the drug loses or reduces its activity when metabolised by CYP2C19 alone or both CYP2C19 and CYP3A4, and when the drug expresses or increases its activity when metabolized by CYP2C19 alone or both CYP2C19 and CYP3A4.

In the case where the drug that loses or reduces its activity when metabolized by CYP2C19 alone or both CYP2C19 and CYP3A4 is used as a drug, when the gastric acidity of the mammalian subject, measured in step (3) is the same as or higher than that of the control mammal, step (4) can determine that the susceptibility of the mammalian subject to the drug is low; and when the gastric acidity of the mammalian subject measured in step (3) is lower than that of the control mammal, step (4) can determine that the susceptibility of the mammalian subject to the drug is high.

In the case where the drug that expresses or increases its activity when metabolized by CYP2C19 alone or both CYP2C19 and CYP3A4 is used as a drug, when the gastric acidity of the mammalian subject measured in step (3) is the same as or higher than that of the control mammal, step (4) can determine that the susceptibility of the mammalian subject to the drug is high; and when the gastric acidity of the mammalian subject measured in step (3) is lower than that of the control mammal, step (4) can determine that the susceptibility of the drug to the subject is low.

### EXAMPLES

The following experimental examples are provided to illustrate the present invention, and are not to limit the scope of the present invention.

### Experimental Example 1

### Evaluation of a Correlation Between the Dose (μmol/kg) of ¹³C—CaCO₃ and the Difference Between ¹³CO₂/¹²CO₂ Concentration Ratios (δ¹³ Values) Before and After Administration [Δ¹³C(‰)=(δ¹³ Value)ₜ−(δ¹³ Value)₀], and Evaluation of a Correlation Between the Dose (μmol/kg) of ¹³C—CaCO₃ and the “Area Under the Δ¹³C(‰)-Time Curve” (AUC)

### (1) Preparation of ¹³C—CaCO₃ Administration Solutions

A ¹³C—CaCO₃ suspension (15 mL) at a concentration of 2500 μmol/4 mL was prepared by adding, in small portions, a 0.5% CMC aqueous solution (sodium carboxymethyl cellulose dissolved in distilled water for injection) to 946.8 mg of ¹³C—CaCO₃ (MW:101, manufactured by Cambridge Isotope Laboratory) while kneading the resulting mixture in a mortar. The obtained ¹³C—CaCO₃ suspension was diluted with the 0.5% CMC aqueous solution to prepare administration solutions at various concentrations (2500, 1000, 500, 200, 100, 20, and 4 μmol/4 mL).

### (2) Experiment

Each of the ¹³C—CaCO₃ suspensions at various concentrations prepared above was forcibly administered orally (4 ml/kg) to fasted rats (male, SD rats: n=3) as experimental animals. Expired air was collected before the administration of the ¹³C—CaCO₃ suspensions (before the ¹³C—CaCO₃ administration) (0 minute) and at each point in time (2, 5, 10, 15, 20, 30, 40, 50, 60, 80, 100, and 120 minutes) after the administration of the ¹³C—CaCO₃ suspensions (after the ¹³C—CaCO₃ administration), and Δ¹³C(‰) was determined with a mass spectrometer for expired air analysis (ABCA: manufactured by SerCon) from the concentration of ¹³CO₂ excreted in the expired air. The Δ¹³C(‰) was determined by measuring ¹³CO₂/¹³CO₂ concentration ratios (δ¹³C values) in the expired air before the ¹³C—CaCO₃ administration (0 minute) and in the expired, air at each point in time for collecting the expired air (t minutes) after the ¹³C—CaCO₃ administration, and calculating Δ¹³C(‰) from the difference [(δ¹³C value)ₜ−(δ¹³C value)₀] between the δ¹³C value [(δ¹³C value)ₜ] at each collection point in time (t) after the ¹³C—CaCO₃ administration and the δ¹³C value [(δ¹³C value)₀] before the ¹³C—CaCO₃ administration (the same applies to the below-described experimental examples).

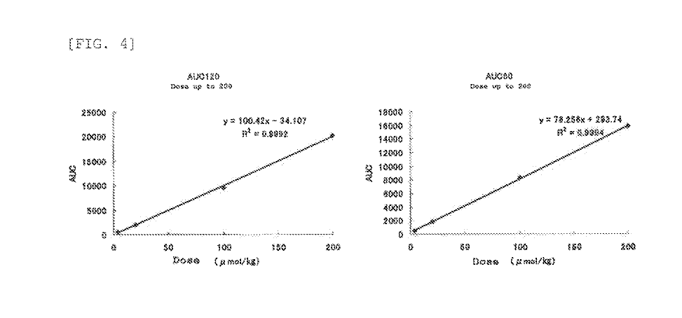

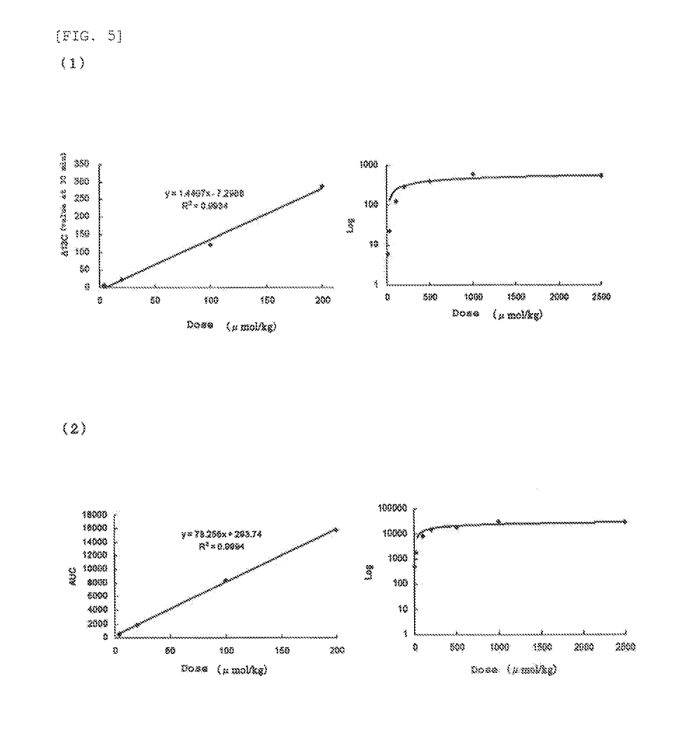

FIG. 2 shows changes in the Δ¹³C(‰) in the expired air measured after each of the ¹³C—CaCO₃ suspensions at various concentrations was administered to the rats. In FIG. 2, the Δ¹³C(‰) in the expired air is plotted on the ordinate, and the expired air collection time (minutes) after the ¹³C—CaCO₃ administration is plotted on the abscissa. As shown in FIG. 2, it was observed that the ¹³C—CaCO₃ was metabolized in the body and excreted as ¹³CO₂ in the expired air. In addition, a saturation phenomenon was observed when the dose exceeded 200 μmol/kg (-▪-).

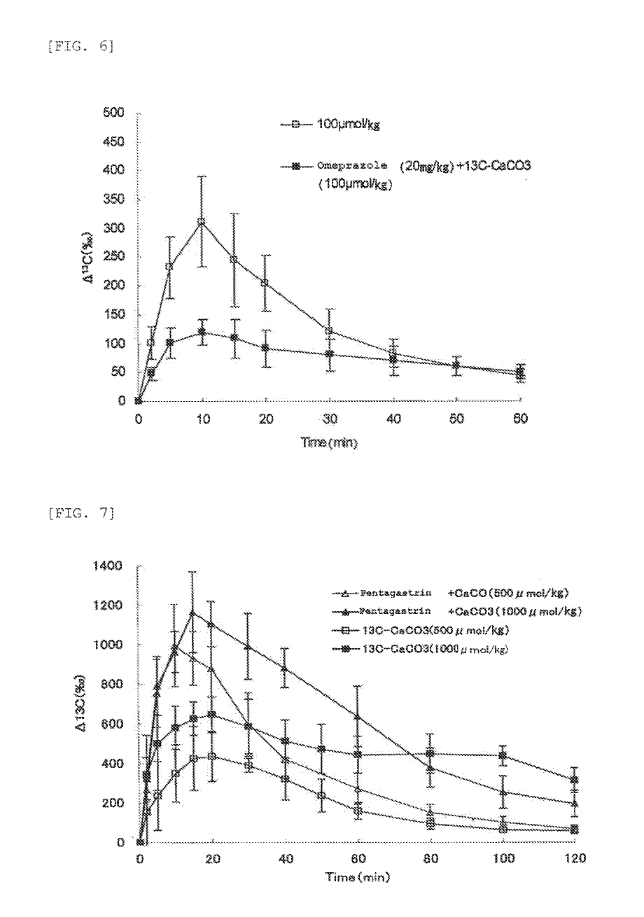

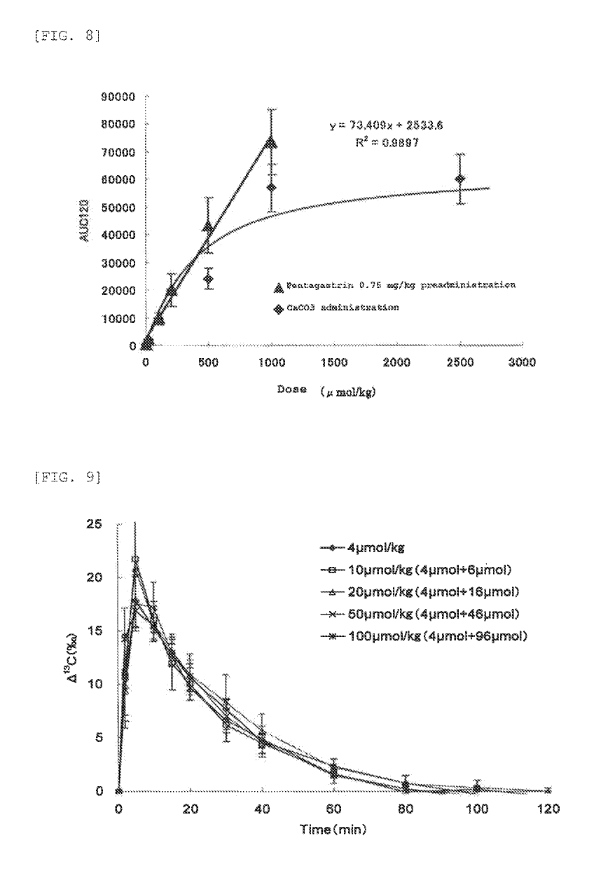

FIG. 3 shows relations between the ¹³C—CaCO₃ suspension dose (μmol/kg) of 4 μmol/kg to 200 μmol/kg and the Δ¹³C(‰) in the expired air at each point, in time for collecting the expired air ((1) 5 minutes after the administration, (2) 10 minutes after the administration, and (3) 15 minutes after the administration). FIG. 3 reveals that the Δ¹³C(‰) in the expired air versus the dose (μmol/kg) up to 200 μmol/kg shows a straight line nearly passing through the origin in all expired air samples, regardless of when the expired air was collected. That is, when the expired air collected at least 5 minutes after the administration of the administration preparation in the dose range of at least not greater than 200 μmol/kg was used as a test sample, a linear correlation was observed between the dose (μmol/kg) of the ¹³C—CaCO₃ and the Δ¹³C(‰) in the expired air (R² values: 0.9 or more).

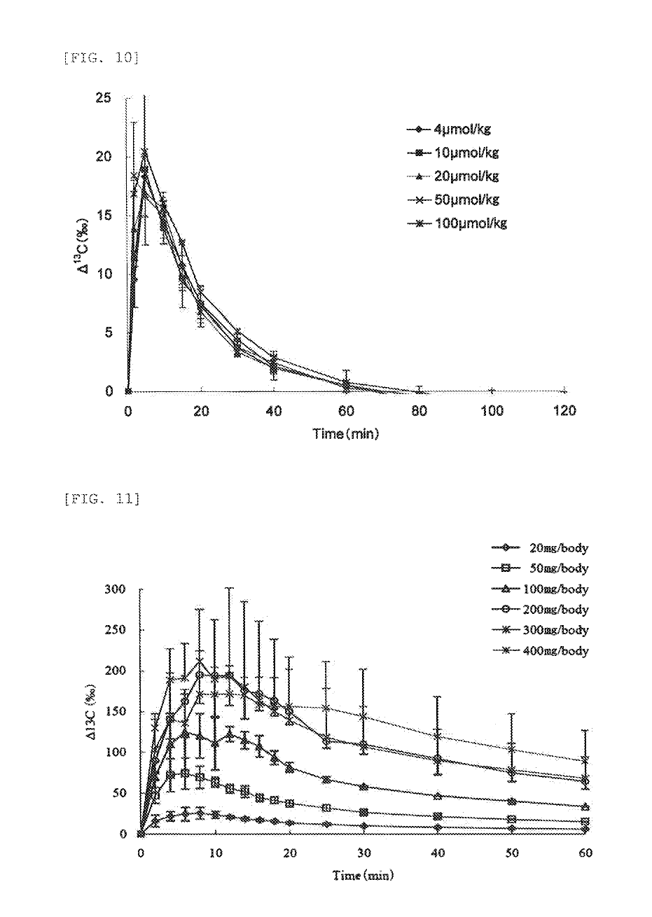

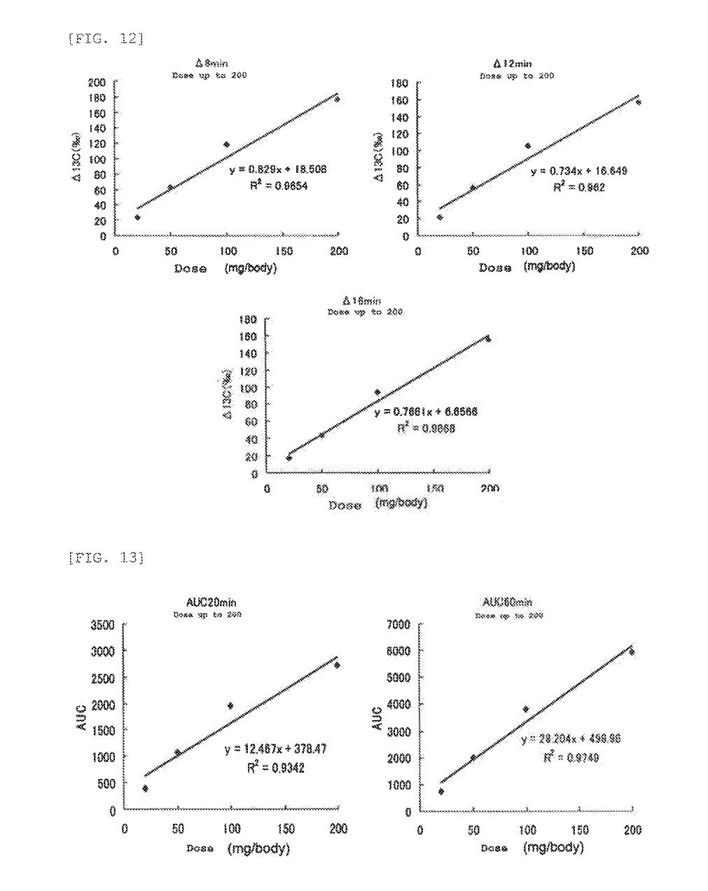

FIG. 4 shows correlations between the ¹³C—CaCO₃ suspension dose (μmol/kg) of 4 μmol/kg to 200 μmol/kg and the “area under the Δ¹³C(‰)-expired air collection time (60 minutes or 120 minutes) curve” (AUC). The graph of the AUC for an expired air collection time of 0 to 60 minutes (AUC60) versus the ¹³C—CaCO₃ dose (4 to 200 μmol/kg) is shown on the right side, whereas the graph of the AUC for an expired air collection time of 0 to 120 minutes (AUC120) versus the ¹³C—CaCO₃ dose (4 to 200 μmol/kg) is shown on the left side. As is clear from these graphs, the AUCs for an expired air collection time of 0 to 60 minutes and for an expired air collection time of 0 to 120 minutes (AUC60 and AUC120) versus the ¹³C—CaCO₃ dose (μmol/kg) up to 200 μmol/kg show straight lines nearly passing through the origin. That is, linear correlations were observed between the AUCs and the ¹³C—CaCO₃ dose in the range of at least not greater than 200 μmol/kg.

The above results indicate that in mammals with normal gastric, acidity, there is a linear correlation between the dose of the ¹³C-labeled carbonate compound in the range of not greater than 200 μmol/kg, and the ¹³CO₂ excretion behavior in the expired air (¹³CO₂ concentration (Δ¹³C(‰)) in the expired air at each point in time for collecting the expired air and the “area under the Δ¹³C(‰)-time curve” (AUC)).

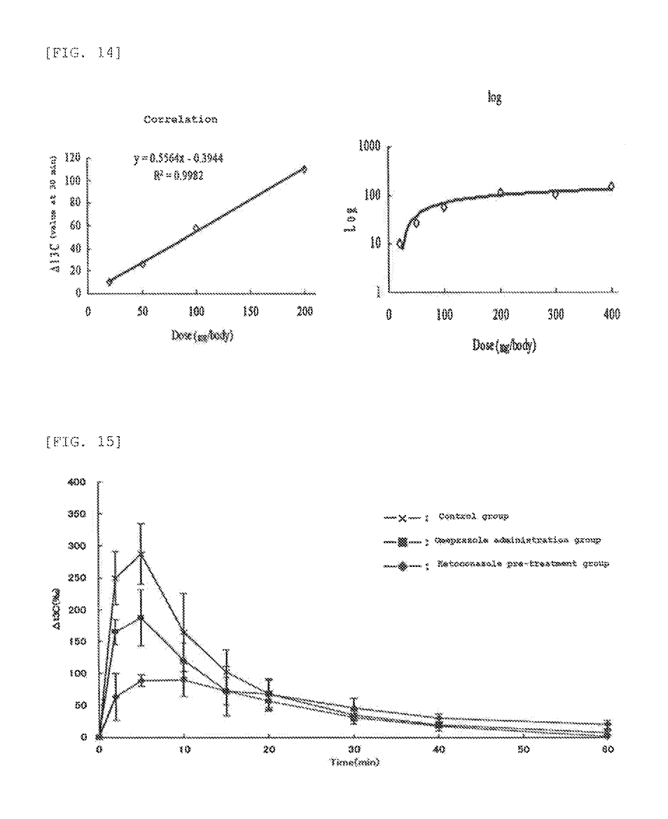

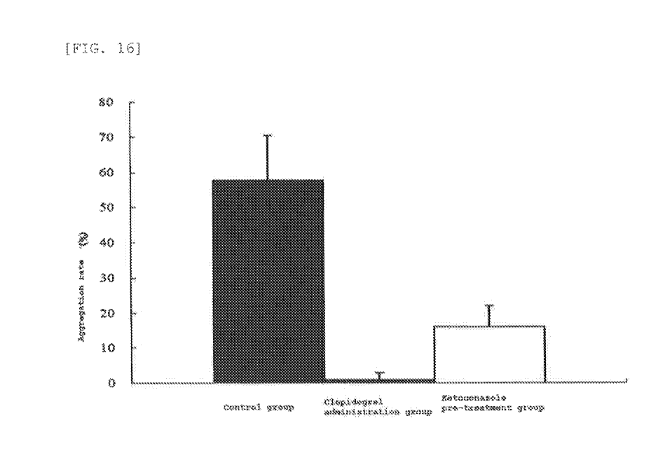

As shown in FIGS. 3 and 4, there is a linear correlation between the ¹³C—CaCO₃ suspension dose (μmol/kg) up to 200 μmol/kg and the Δ¹³C(‰), and there is a linear correlation between the ¹³C—CaCO₃ suspension dose (μmol/kg) up to 200 μmol/kg and the “area under the Δ¹³C(‰)-time curve” (AUC). FIG. 5 (1) shows a relation between the dose of 4 to 2500 μmol/kg and the Δ¹³C(‰) at an expired air collection time of 30 minutes. In FIG. 5(1), a relation between the ¹³C—CaCO₃ dose (4 to 200 μmol/kg) and the Δ¹³C(‰) is shown on the left side, whereas the relation between the ¹³C—CaCO₃ dose (4 to 2500 μmol/kg) and the Δ¹³C(‰) is shown on the right side. The results reveal that when the ¹³C—CaCO₃ dose is not greater than a certain amount, there is a linear correlation between the dose and the Δ¹³C(‰); however, when the dose exceeds the certain amount, the Δ¹³C(‰) value becomes constant (plateau), as shown in FIG. 1 (1).

FIG. 5 (2) shows a relation between the dose of 4 to 2500 μmol/kg and AUC for an expired, air collection time of 0 to 120 minutes. In FIG. 5 (2), a relation between the ¹³C—CaCO₃ dose (4 to 200 μmol/kg) and the AUC is shown on the left side (the same as the graph on the right side of FIG. 4), whereas the relation between the ¹³C—CaCO₃ dose (4 to 2500 μmol/kg) and the AUC is shown on the right side. The results reveal that as in the above Δ¹³C(‰), when the ¹³C—CaCO₃ dose is not greater than a certain amount, there is a linear correlation between the dose and the AUC; however, when the dose exceeds the certain amount, the AUC value becomes constant (plateau), as shown in FIG. 1 (2).

### Experimental Example 2

### Measurement of Gastric Acid Secretion Decrease

### (1) Preparation of a Proton Pump Inhibitor (PPI: Omeprazole) Administration Solution

To 1 vial of Omepral Injection 20 (omeprazole 20 mg/vial, AstraZeneca K.K.) that, has the action of suppressing gastric acid secretion, 1 mL of physiological saline was added so that the concentration was 20 mg/ml, and the resulting mixture was used as a PPI administration solution.

### (2) Experiment

Fasted rats (male, SD rats) were used as experimental animals.

To Group 1 (control group: normal rats, n3), the ¹³C—CaCO₃ suspension (100 μmol/4 mL) prepared in Experimental Example 1 was orally administered (4 mL/kg). To Group 2 (model group with decreased gastric acidity: rats given PPI, n=3), the PPI administration solution (20 mg/mL) was intravenously administered (1 mL/kg; to produce animal models with suppressed gastric acid secretion, and the ¹³C—CaCO₃ suspension (100 μmol/4 mL) was orally administered (4 mL/kg) 4 hours after the administration of the PPI administration solution.

In the rats in each group, expired air was collected before the administration of the ¹³C—CaCO₃ suspension (0 minute) and at each point in time (2, 5, 10, 15, 20, 30, 40, 50, and 60 minutes) after the administration of the ¹³C—CaCO₃ suspension; and Δ¹³C(a) was measured with a mass spectrometer for expired air analysis (ABCA: manufactured by SerCon) from, the concentration of ¹³CO₂ excreted in the expired air.

FIG. 6 shows the results. In FIG. 6, -□- and -▪- respectively show changes in the Δ¹³C(‰) in the expired air measured in Group 1 (control group) and Group 2 (model group with decreased gastric acidity) over time.

FIG. 6 reveals that the Δ¹³C(‰) in the expired air of the rats whose gastric acid secretion had been suppressed by the administration of the PPI (model group with decreased gastric acidity: -▪-) was significantly lower than the Δ¹³C(‰) in the expired air of the normal rats (control group: -□-).

The above results indicate that even, in the case of mammals with decreased gastric acidity due to suppression of gastric acid secretion, there is a linear correlation (linearity) between the dose of the ¹³C-labeled carbonate compound in the range of at least not greater than 100 μmol/kg, and the ¹³CO₂ excretion behavior in the expired air ¹³CO₂ concentration (Δ¹³C(‰)) in the expired air at each point in time for collecting the expired air and “area under the Δ¹³C(‰) -time curve” (AUC)).

### Experimental Example 3

### Measurement of Gastric Acid Secretion Increase

### (1) Preparation of a Pentagastrin Administration Solution

DMSO and 2 ml of physiological saline were added to 7.5 mg of pentagastrin (SIGMA) that has the action of stimulating and increasing gastric acid secretion so that the total amount was 10 ml, to prepare a pentagastrin administration solution at a concentration of 0.75 mg/ml.

### (2) Experiment

Fasted rats (male, SD rats) were used as experimental animals.

Two groups, Group 1 (normal rats, n3) and Group 2 (normal rats, n=3), were provided as control groups. The ¹³C—CaCO₃ suspension prepared, in Experimental Example 1 was orally administered to Group 1 and Group 2 in an amount of 500 μmol/kg and 1000 μmol/kg, respectively. As model groups with increased gastric acidity, two groups, Group 3 (n=3) and Group 4 (n=3), were provided. The pentagastrin solution (0.75 mg/mL) was intravenously administered (1 mL/kg) to Group 3 and Group 4 to increase gastric acid secretion (production of animal models with increased gastric acid secretion); and the ¹³C—CaCO₃ suspension was orally administered to Group 3 and Group 4 in an amount of 500 μmol/kg and 1000 μmol/kg, respectively, 1 hour after the administration of the pentagastrin.

In the rats in each group, expired air was collected before the administration of the ¹³C—CaCO₃ suspension (0 minute) and at each point in time (2, 5, 10, 15, 20, 30, 40, 50, 60, 80, 100, and 120 minutes) after the administration of the ¹³C—CaCO₃ suspension, and Δ¹³C(‰) was measured, with a mass spectrometer for expired air analysis (ABCA; manufactured by SerCon) from the concentration of ¹³CO₂ excreted in the expired air.

FIG. 7 shows the results. In FIG. 7, -□- and -▪- show changes in the ¹³CO₂ concentration (Δ¹³C(‰)) in the expired air-measured in the control groups Group 1 (¹³C—CaCO₃ suspension (500 μmol/kg)-administration group) and Group 2 (¹³C—CaCO₃ suspension (1000 μmol/kg)-administration group), respectively; -Δ- and -▴- show changes in the ¹³CO₂ concentration (Δ¹³C(‰)) in the expired air measured in the model groups with increased gastric acidity Group 3 (pentagastrin+¹³C—CaCO₃ suspension (500 μmol/kg)-administration group) and Group 4 (pentagastrin+¹³C—CaCO₃ suspension (1000 μmol/kg)-administration group), respectively.

FIG. 7 reveals that the ¹³CO₂ concentration (Δ¹³C(‰)) in the expired air of the rats whose gastric acid secretion had been increased by the administration of the pentagastrin (model groups with increased gastric acidity: -Δ- and -▴-) was significantly high compared to the ¹³CO₂ concentration (Δ¹³C(‰)) in the expired air of the normal rats (control groups: -□- and -▪-).

FIG. 8 shows a comparison of correlations between the ¹³C—CaCO₃ dose (μmol/kg) of 4 to 2500 μmol/kg and the “area under the Δ¹³C(‰) -time curve” for an expired air collection time of 0 to 120 minutes (AUC120) in the control group (-♦-) and the model group with increased gastric acidity (-▴-). Note that, the relation observed between the ¹³C—CaCO₃ dose and the AUC is also found between the ¹³C—CaCO₃ dose and the Δ¹³C(‰).

As is clear from the results, in the normal model (control group) Group 1 (-♦-), the plots are linear on a straight line passing through the origin in the dose up to 200 μmol/kg, indicating that there is a linear correlation between the dose and, the AUC; however, when the dose exceeds 200 μmol/kg, a plateau phenomenon was observed. On the other hand, in the model with increased, gastric acidity Group 2 (-▴-), the plots are on a straight line passing through the origin in the dose of at least up to 1000 μmol/kg, indicating that there is a linear correlation between the dose and, the AUC.

The above results indicate that even in the case of mammals with increased gastric acidity due to increased gastric acid secretion, there is a linear correlation (linearity) between the dose of the ¹³C-labeled carbonate compound in the range of at least not greater than 1000 μmol/kg, and the ¹³CO₂ excretion behavior in the expired air (¹³CO₂ concentration (Δ¹³C(‰)) in the expired air at each point in time for collecting the expired air and the “area under the Δ¹³C(‰) -time curve” (AUC). The results also snow that in the model with increased, gastric acidity, the range of dose (μg/kg) in which a linear correlation (linearity) was observed between the dose and the Δ¹³C(‰), and the range of dose (μg/kg) in which a linear correlation (linearity) was observed between the dose and the AUC were broader than those in the normal model, i.e., a plateau phenomenon was seen in the model with increased, gastric acidity at a dose higher than that in the normal model (see FIG. 1).

From the results of Experimental Examples 2 and 3, the possibility was confirmed that gastric acidity can be quantitatively measured by an expiration test that measures ¹³CO₂ concentration excreted in expired air after administration of ¹³C—CaCO₃; and that decrease or increase of gastric acid, secretion can be evaluated from the dose and ¹³CO₂ excretion behavior in the expired air (¹³CO₂ concentration (Δ¹³C(‰)) in the expired air at each point in time for collecting the expired air and “area under the Δ¹³C(‰) -time curve” (AUC)).

### Experimental Example 4

### Administration of a ¹²C—CaCO₃ and ¹³C—CaCO₃ Mixture

(1) Preparation of Administration Solutions of a ¹²C—CaCO₃ and ¹³C—CaCO₃ Mixture (10, 20, 50, 100 μmol/4 mL)

A ¹³C—CaCO₃ suspension (20 mL) at a concentration of 4 μmol/2 mL was prepared by adding, in small portions, a 0.5% CMC aqueous solution (sodium carboxymethyl cellulose dissolved in distilled water for injection) to 10.1 mg of ¹³C—CaCO₃ (MW:101, manufactured by Cambridge Isotope Laboratory) while kneading the resulting mixture in a mortar.

¹²C—CaCO₃ suspensions at concentrations of 6, 16, 46, and 96 μmol/2 mL were prepared by adding, in small portions, the 0.5% CMC solution to calcium carbonate (MW:100, Wako Pure Chemical Industries, Ltd.) while kneading the resulting mixture in a mortar.

The thus-obtained suspensions were mixed in equal amounts to prepare administration solutions of a ¹²C—CaCO₃ and ¹³C—CaCO₃ mixture (hereinafter, simply referred to as “mixture administration solutions”) at concentrations of 10, 20, 50, and 100 μmol/4 mL, respectively, A ¹³C—CaCO₃ solution at a concentration of 4 μmol/4 mL was prepared by mixing the ¹³C—CaCO₃ suspension (4 μmol/2 mL) and the 0.5% CMC solution in equal amounts,

### (2) Experiment

Each of the mixture administration solutions at various concentrations prepared above was forcibly administered orally (4 ml/kg) to fasted rats (male, SD rats: n=3) as experimental animals. Expired air was collected before the administration of the mixture administration solutions (0 minute) and at each point in time (2, 5, 10, 15, 20, 30, 40, 60, 80, 100, and 120 minutes) after the administration of the mixture administration solutions, and Δ¹³C(‰) was determined with a mass spectrometer for expired air analysis (ABCA; manufactured by SerCon) from the concentration of ¹³CO₂ excreted in the expired air.

FIG. 9 shows the results. FIG. 9 shows changes in the Δ¹³C(‰) in the expired air after each, of the mixture administration solutions was administered to the rats. As is clear from this graph, it is shown that even in the case where ¹²C—CaCO₃ is added to ¹³C—CaCO₃ and the resulting mixture is administered, the behavior of the Δ¹³C(‰) in the expired air is not affected by the ¹²C—CaCO₃.

### Experimental Example 5

### Administration of ¹³C—CaCO₃ and Sodium Acetate Mixtures

(1) Preparation of ¹³C—CaCO₃ and Sodium, Acetate Mixtures (4, 10, 20, 50, and 100 μmol/4 mL)

A ¹³C—CaCO₃ suspension (20 mL) at a concentration of 4 μmol/2 mL was prepared by adding, in small portions, a 0.5% CMC aqueous solution (sodium carboxymethyl cellulose dissolved in distilled water for injection) to 10.1 mg of ¹³C—CaCO₃ (MW:101, manufactured by Cambridge Isotope Laboratory) while kneading the resulting mixture in a mortar.

Sodium acetate solutions at predetermined concentrations (6, 16, 46, and 96 μmol/2 mL) were prepared by adding the 0.5% CMC aqueous solution to sodium acetate (MW:82, Wako Pure Chemical Industries, Ltd.).

The thus-obtained solutions were mixed in equal amounts to prepare mixtures of the ¹³C—CaCO₃ and the sodium acetate (10, 20, 50, and 100 μmol/4 mL, respectively). A ¹³C—CaCO₃ solution at a concentration of 4 μmol/4 mL was prepared by mixing the ¹²C—CaCO₃ suspension (4 μmol/2 mL) and the 0.5% CMC solution in equal amounts.

### (2) Experiment

Each of the ¹³C—CaCO₃ and sodium acetate mixtures at various concentrations (4, 10, 20, 50, and 100 μmol/4 ml; (hereinafter, simply referred to as “mixtures”) was forcibly administered orally (4 mL/kg) to fasted rats (male, SD rats: n=3) as experimental animals. Expired air was collected before the administration of the mixtures (0 minute) and at each point in time (2, 5, 10, 15, 20, 30, 40, 60, 80, 100, and 120 minutes) after the administration of the mixtures, and Δ¹³C(‰) was determined with a mass spectrometer for expired air analysis (ABCA: manufactured by SerCon) from the concentration of ¹³CO₂ excreted in the expired air.

FIG. 10 shows the results. FIG. 10 shows changes in the Δ¹³C(‰) in the expired, air after each of the mixtures was administered, to the rats. As is clear from FIG. 10, it is shown that even in the case where an alkaline material such as sodium acetate, which does not contain ¹³C, is added to −¹³C—CaCO₃ and the resulting mixture is administered, there is no influence on the behavior of the ¹³CO₂ concentrated on in the expired air. That is, it is revealed that even in the case where an alkaline material is mixed with ¹³C—CaCO₃, there is no influence on an expiration test that measures ¹³CO₂ concentration excreted in expired air after administration of ¹³C—CaCO₃.

As shown in Experimental Examples 4 and 5, when the ¹²C-carbonate compound or sodium, acetate was mixed with the ¹³C-labeled carbonate compound, there was no influence on the behavior of ¹³CO₂. This fact indicates that the method of the present invention can be performed using a smaller amount of the ¹³C-labeled carbonate compound by adding the ¹²C-carbonate compound or sodium acetate. That is, the amount of the ¹³C-labeled carbonate compound can be reduced, thereby reducing the cost.

In Experimental Examples 1 to 5 described above, used as mammals were rats, which are experimental animals widely used as mammals with a respiratory system and digestive system (in particular, gastric acid secretion system) that have functions similar to those of a human respiratory system and digestive system. Rats are hitherto used for screening stimulants of gastric acid secretion or gastric acid secretion inhibitors. As shown in Experimental Example 6 below, all of the aforementioned results can be easily extrapolated to humans, and similar results can also be obtained in the case where the above experiments are carried out for humans.

### Experimental Example 6

### Evaluation of Correlations Between the Dose (mg/body) of ¹³C—CaCO₃ and the Δ¹³C(‰), and Between the Dose (mg/body) of ¹³C—CaCO₃ and the “Area Under the Δ¹³C(‰)-Time Curve” (AUC)” (Humans)

The same experiment as in Experimental Example 1 was conducted for humans (n=3) with normal gastric acidity.

### (1) Preparation of ¹³C—CaCO₃ Administration Suspensions

Administration suspensions at various concentrations (400, 300, 200, 100, 50, and 20 mg/50 mL) were prepared by adding, in small portions, a 0.5% CMC aqueous solution (sodium carboxymethyl cellulose dissolved in distilled water for injection) to ¹³C—CaCO₃ (MW: 101, manufactured by Cambridge Isotope Laboratory) while kneading the resulting mixture in a mortar.

### (2) Experiment

To human subjects (n=3) who had abstained from food or drink from 21:00 the day before administration, each of the ¹³C—CaCO₃ suspensions at various concentrations prepared above was orally administered at 8:30 the next morning (50 mL/body). Expired air was collected before the administration of the ¹³C—CaCO₃ suspensions (before the ¹³C—CaCO₃ administration) (0 minute) and at each point. In time (2, 4, 6, 8, 10, 12, 14, 16, 18, 20, 25, 30, 40, 50, and 60 minutes) after the administration of the ¹³C—CaCO₃ suspensions (after the ¹³C—CaCO₃ administration), and Δ¹³C(‰) was determined with a mass spectrometer for expired air analysis (ABCA: manufactured by SerCon) from the concentration of ¹³CO₂ excreted in the expired air.

FIG. 11 shows changes in the Δ¹³C(‰) in the expired air measured after each of the ¹³C—CaCO₃ suspensions at various concentrations (400, 300, 200, 100, 50, and 20 mg/body) was administered to the human subjects. In FIG. 11, the Δ¹³C(‰) in the expired air is plotted on the ordinate, and the expired air collection time (minutes) after the ¹³C—CaCO₃ administration is plotted on the abscissa. As shown in FIG. 11, it was observed that the ¹³C—CaCO₃ was metabolized in the body and excreted as ¹³CO₂ in the expired air. In addition, a saturation phenomenon was observed when the dose exceeds 200 mg/body (33 μmol/kg).

FIG. 12 shows relations between the ¹³C—CaCO₃ suspension dose (mg/body) of 20 mg/body to 200 mg/body and the Δ¹³C(‰) in the expired air at each point in time for collecting the expired air ((1) 8 minutes after the administration, (2) 12 minutes after the administration, and (3) 16 minutes after the administration). FIG. 12 reveals that the Δ¹³C(‰) in the expired air versus the dose (mg/body) up to 200 mg/body shows a straight line nearly passing through the origin in all expired air samples, regardless of when the expired air was collected. That is, when the expired air collected at least 8 minutes after the administration of the administration preparation in the dose range of at least not greater than 200 mg/body was used as a test sample, a linear-correlation was observed between the ¹³C—CaCO₃ dose (mg/body) and the Δ¹³C(‰) in the expired air (R² values: 0.9 or more).

FIG. 13 shows correlations between the ¹³C—CaCO₃ suspension dose (mg/body) of 20 mg/body to 200 mg/body and the “area under the Δ¹³C(‰)-expired air collection time (20 minutes or 60 minutes) curve” (AUC). The graph of the AUC for an expired air collection time of 0 to 20 minutes (AUC20) versus the ¹³C—CaCO₃ dose (20 to 200 mg/body) is shown on the left side, whereas the graph of the AUC for an expired air collection time of 0 to 60 minutes (ADC60) versus the ¹³C—CaCO₃ dose (20 to 200 mg/body) is shown on the right side. As is clear from these graphs, the AUCs for an expired air collection time of 0 to 20 minutes and for an expired air collection time of 0 to 60 minutes (AUC20 and AUC60) versus the ¹³C—CaCO₃ dose (mg/body) up to 200 mg/body show straight lines nearly passing through the origin. That is, linear correlations were observed between the AUCs and the ¹³C—CaCO₃ dose of at least not greater than 200 mg/body.

The above results indicate that in humans with normal gastric acidity, there is a linear correlation between the dose of the ¹³C-labeled carbonate compound in the range of not greater than 200 mg/body and the ¹³CO₂ excretion behavior in the expired air (¹³CO₂ concentration (Δ¹³C(‰)) in the expired air at each point in time for collecting the expired air and the “area under the Δ¹³C(‰)-time curve” (AUC)).

As shown in FIGS. 12 and 13, there is a linear correlation between the ¹³C—CaCO₃ suspension dose (mg/body) up to 200 mg/body and the Δ¹³C(‰), and there is a linear correlation between the ¹³C—CaCO₃ suspension dose (mg/body) up to 200 mg/body and the “area under the Δ¹³C(‰)-time curve” (ADC). FIG. 14 shows a relation between the dose of 20 to 400 mg/body and the Δ¹³C(‰) at an expired air collection time of 30 minutes. The results reveal that when the ¹³C—CaCO₃ dose is not greater than a certain amount (here, not greater than 200 mg/body), there is a linear correlation between the dose and the Δ¹³C(‰); however, when the dose exceeds the certain amount, the Δ¹³C(‰) value becomes constant (plateau), as shown in FIG. 1 (1).

### Experimental Example 7

### A Correlation Between the Enzyme Activity of CYP2C19 and CYP3A4 and the Effect OF Omeprazole

Although omeprazole, which is a proton pump inhibitor (PPI), is mainly metabolised by hepatic metabolism enzyme CYP2C19, it is also metabolized by CYP3A4 as a bypass pathway. Thus, the effect of omeprazole depends on the metabolic capacity of CYP2C19 and CYP3A4 in a subject. By using the method for measuring gastric acidity of the present invention, a correlation between the enzyme activity of CYP2C19 and CYP3A4 and the effect of omeprazole was evaluated using a CYP2C19 and CYP3A4 inhibitor (ketoconazole).

### (1) Preparation of Test Samples

(i) ketoconazole (CYP2C19 and CYP3A4 inhibitor: A ketoconazole suspension at a concentration of 50 mg/4 mL (23.54 μmol/4 mL) was prepared by adding, in small portions, a 0.5% CMC aqueous solution to ketoconazole while kneading the resulting mixture in a mortar.
(ii) Omeprazole: Omeprazole was prepared by dissolving omepral for injection in physiological saline so that the concentration was 3 mg/ml.
(iii) ¹³C—CaCO₃ suspension: A ¹³C—CaCO₃ suspension at a concentration of 100 μmol/4 mL was prepared by adding, in small portions, a 0.5% CMC aqueous solution to ¹³C—CaCO₃ while kneading the resulting mixture in a mortar,

### (2) Experiment

Fasted rats (7 weeks old, female, SD rats) were used as experimental animals. The rats were divided into an omeprazole administration group (n=3), a ketoconazole pre-treatment group (n=3), and a control group (n=3).

To the omeprazole administration group, a single dose of the omeprazole (3 mg/kg) was intravenously administered, and the ¹³C—CaCO₃ suspension (100 μmol/4 mL) was orally administered at a dose of 4 mL/kg 2 hours after the administration. Expired air was then collected. To the ketoconazole pre-treatment group, a single dose of the ketoconazole (50 mg/kg) was orally administered in advance, a single dose of the omeprazole (3 mg/kg) was intravenously administered 30 minutes after the administration, and the ¹³C—CaCO₃ suspension (100 μmol/4 mL) was orally administered at a dose of 4 mL/kg 2 hours after the administration of the omeprazole. Expired air was then collected. To the control group (rats that had not been treated), only the ¹³C—CaCO₃ suspension (100 μmol/4 mL) was orally administered at a dose of 4 mL/kg, and expired air was collected.

The above expired air was used as test samples, and Δ¹³C(‰) was measured with a mass spectrometer for expired air analysis (ABCA; manufactured by SerCon) from the concentration of ¹³CO₂ excreted in the expired air.

FIG. 15 shows the results. In FIG. 15, -x- indicates change in the ¹³CO₂ concentration (Δ¹³C(‰)) in the expired air measured in the control group; -▪- indicates change in the ¹³CO₂ concentration (Δ¹³C(‰)) in the expired air measured in the omeprazole administration group; and -♦- indicates change in the ¹³CO₂ concentration (Δ¹³C(‰)) in the expired air measured in the ketoconazole pre-treatment group.

As shown, in FIG. 15, in the omeprazole administration group, gastric acid secretion was suppressed, and then Δ¹³C(‰)) in the expired air after administering the ¹³C—CaCO₃ was decreased, compared to the control group. Further, by the ketoconazole pre-treatment, Δ¹³C(‰) in the expired air after administering the ¹³C—CaCO₃ was decreased. It is believed that this phenomenon indicates that metabolism of the omeprazole in the body was inhibited by the CYP2C19 and CTP3A4 inhibitor (ketoconazole) to enhance the action of suppressing gastric acid secretion, thereby decreasing gastric acidity.

This experiment confirmed chat by using the method for measuring gastric acidity of the present invention, the efficacy (effect; of a drug metabolized by CYP2C19 or by CYP2C19 and CYP3A4 in each subject can be evaluated. The effect of omeprazole and that: of the below-described clopidogrel vary between individuals. It is suggested that this is partially attributable to involvement of polymorphisms of the drug metabolising enzyme CYP2C19 gene or/and CYP3A4 gene. Thus, by using the method for measuring gastric acidity of the present invention, the enzyme activity (metabolic capacity; of CYP2C19 alone or both CYP2C19 and CYP3A4 and the presence of genetic polymorphisms of CYP2C19or/and CYP3A4 in each subject can be evaluated; and the effect of a drug relating to the enzyme activity (metabolic capacity) of CYP2C19 alone or both CYP2C19 and CTP3A4 on a subject (susceptibility of a subject to the drug) can also be evaluated.

### Reference Example 1

### A Correlation Between the Enzyme Activity of CYP2C19 and the Effect of Clopidogrel

Clopidogrel, an antithrombotic drug, is converted by CYP2C19 and CYP3A4 into an active metabolite, which binds to the P2Y12 receptor expressed on the surface of platelets to inhibit platelet aggregation. Thus, the effect of clopidogrel depends on the metabolic capacity of CYP2C19 and CYP3A4 in a subject. By using the method for measuring gastric acidity of the present invention, a correlation between the activity of CYP2C19 and CYP3A4, and the effect of clopidogrel was evaluated, using a CYP2C19 and CYP3A4 inhibitor (ketoconazole).

### (1) Preparation of Test Samples

(i) Ketoconazole (CYP2C inhibitor): A ketoconazole suspension at a concentration of 50 mg/4 mL (23.54 μmol/4 mL) was prepared by adding, in small portions, a 0.5% CMC aqueous solution to ketoconazole while kneading the resulting mixture in a mortar.
(ii) Clopdogrel: Clopidogrel was prepared by dissolving clopidogrel in a 0.5% CMC aqueous solution so that the concentration was 10 mg/mL.

### (2) Experiment

Fasted rats (7 weeks old, female, SD rats) were used as experimental animals. The rats were divided into a clopidogrel administration group (n=3), a ketoconazole pre-treatment group (n=3), and a control group (n=3).

To the clopidogrel administration group, a single dose of the clopidogrel (10 mg/kg) was orally administered, and brood was collected 2 hours after the administration. To the ketoconazole pre-treatment group, a single dose of the ketoconazole (50 mg/kg) was orally administered in advance, a single dose of the clopidogrel (10 mg/kg) was orally administered 30 minutes after the administration, and blood was collected 2 hours after the administration of the clopidogrel,

Using the collected platelet-rich plasma, platelet aggregation by ADP stimulation was measured with a multifunctional platelet aggregation measurement device (manufactured by MC Medical, Inc.).

FIG. 16 shows the results. As is clear from these results, the platelet aggregation inhibitory action of the clopidogrel was reduced by the CYP2C19 and CYP3A4 inhibitor ketoconazole. It is believed that the metabolic activation of the clopidogrel in the body was inhibited by the CYP2C19 and CYP3A4 inhibitor (ketoconazole), resulting in the reduction in the platelet aggregation inhibitory action.

From these results and the results in Experimental Example 6, it is believed that the efficacy (effect) of clopidogrel in each subject can be evaluated by using the method for measuring gastric acidity of the present invention. Further, it is also believed that by using the method for measuring gastric acidity of the present invention, the effect of clopidogrel relating to the enzyme activity (metabolic activity) of CYP2C13 and CYP3A4 on a subject (susceptibility of a subject to clopidogrel) can be evaluated.

1. A method for measuring gastric acidity of a mammal comprising the steps of:
(1) using, as a test sample, expired air of a mammalian subject excreted at any point in time within 30 minutes after oral administration of a predetermined amount of a ¹³C-labeled carbonate compound, measuring behavior of ¹³CO₂ in the expired air;
(2) comparing the behavior of ¹³CO₂ (measured ¹³CO₂ behavior) obtained in step (1) with the behavior of corresponding ¹³CO₂ (reference ¹³CO₂ behavior) that has been obtained beforehand in a control mammal; and
(3) determining the gastric acidity of the mammalian subject based on a difference between the reference ¹³CO₂ behavior and the measured ¹³CO₂ behavior obtained in step (2).

2. The method according to claim 1, wherein the behavior of ¹³CO₂ is Δ¹³C(‰) obtained from expired air of a mammalian subject collected at any point in time within 30 minutes after oral administration of a ¹³C-labeled carbonate compound.

3. The method according to claim 1, wherein the predetermined amount is 10 mg to 5 g.

4. The method according to claim 1, wherein the ¹³C-labeled carbonate compound is at least one carbonate compound selected from the group consisting of alkali metal carbonates, alkaline earth metal carbonates, ammonium carbonate, alkali metal hydrogencarbonates, and ammonium hydrogencarbonate.

5. The method according to claim 2,
wherein determination step (3) is a step of determining that the gastric acidity of a mammalian subject is the same as or higher than the gastric acidity of a control mammal when measured ¹³CO₂ behavior and reference ¹³CO₂ behavior are the same, or determination step (3) is a step of determining that the gastric acidity of a mammalian subject is lower than the gastric acidity of a control mammal when measured ¹³CO₂ behavior is lower than reference ¹³CO₂ behavior.

6. (canceled)

7. The method according to claim 2, wherein the control mammal used in comparing step (2) is a mammal having normal gastric acidity.

8. A method for measuring an effect of a gastric acid reducer on a mammal, the method comprising the following steps (1) to (4):
(1) using, as a test sample, expired air of a mammalian subject excreted at any point in time within 30 minutes after oral administration of a predetermined amount of a ¹³C-labeled carbonate compound, the oral administration being performed after administration of a gastric acid reducer, measuring behavior of ¹³CO₂ excreted in the expired air,
(2) comparing the behavior of ¹³CO₂ (measured ¹³CO₂ behavior) obtained in step (1) with the behavior of corresponding ¹³CO₂ (reference ¹³CO₂ behavior) measured in a mammal (control mammal) to which a predetermined amount of a ¹³C-labeled carbonate compound has been orally administered beforehand without administering the gastric acid reducer;
(3) determining gastric acidity of the mammalian subject based on a difference between the reference ¹³CO₂ behavior and the measured ¹³CO₂ behavior obtained above; and
(4) determining the effect of the gastric acid reducer on the mammalian subject using the gastric acidity of the mammalian subject obtained above as an index.

9. The method according to claim 8, wherein the behavior of ¹³CO₂ is Δ¹³C(‰) obtained from expired air of a mammalian subject collected at any point in time within 30 minutes after oral administration of a ¹³C-labeled carbonate compound.

10. The method according to claim 8, wherein the predetermined amount is 10 mg to 5 g.

11. The method according to claim 8, wherein the ¹³C-labeled carbonate compound is at least one carbonate compound selected from the group consisting of alkali metal carbonates, alkaline earth metal carbonates, ammonium carbonate, alkali metal hydrogencarbonates, and ammonium hydrogencarbonate.

12. The method according to claim 8, wherein step (3) is a step of determining that the gastric acidity of the mammalian subject is the same as or higher than the gastric acidity of the control mammal when the measured ¹³CO₂ behavior and the reference ¹³CO₂ behavior are the same, or that the gastric acidity of the mammalian subject is lower than the gastric acidity of the control mammal when the measured ¹³CO₂ behavior is lower than the reference ¹³CO₂ behavior.

13. The method according to claim 12, wherein step (4) is a step of determining that the administered gastric acid reducer has no effect on the mammalian subject when the gastric acidity of the mammalian subject measured in step (3) is the same as or higher than the gastric acidity of the control mammal; or that the administered gastric acid reducer has an effect on the mammalian subject when the gastric acidity of the mammalian subject measured in step (3) is lower than the gastric acidity of the control mammal.

14. A method for evaluating enzyme activity (metabolic capacity) of CYP2C19 alone or both CYP2C19 and CYP3A4 in a mammalian subject, effect of a drug metabolized by CYP2C19 alone or both CYP2C19 and CYP3A4 on the mammalian subject, or/and susceptibility of the mammalian subject to the drug, the method comprising the following steps (1) to (4):
(1) using, as a test sample, expired air of a mammalian subject excreted at any point in time within 30 minutes after oral administration of a predetermined amount of a ¹³C-labeled carbonate compound, the oral administration being performed after administration of omeprazole or lansoprazole, measuring behavior of ¹³CO₂ excreted in the expired air,
(2) comparing the behavior of ¹³CO₂ (measured ¹³CO₂ behavior) obtained in step (1) with behavior of corresponding ¹³CO₂ (reference ¹³CO₂ behavior) measured in a mammal (control mammal) to which a predetermined amount of a ¹³C-labeled carbonate compound has been orally administered beforehand without administering omeprazole and lansoprazole;
(3) determining the gastric acidity of the mammalian subject based on a difference between the reference ¹³CO₂ behavior and the measured ¹³CO₂ behavior obtained above; and
(4) determining the enzyme activity (metabolic capacity) of CYP2C19 alone or both CYP2C19 and CYP3A4 in the mammalian subject, effect of a drug metabolized by CYP2C19 alone or both CYP2C19 and CYP3A4 on the mammalian subject, or/and susceptibility of the mammalian subject to the drug, using the gastric acidity of the mammalian subject obtained above as an index.

15. The method according to claim 14, wherein the behavior of ¹³CO₂ is Δ¹³C(‰) obtained from expired air of a mammalian subject collected at any point in time within 30 minutes after oral administration of a ¹³C-labeled carbonate compound.

16. The method according to claim 14, wherein the predetermined amount is 10 mg to 5 g.

17. The method according to claim 14, wherein the ¹³C-labeled carbonate compound is at least one carbonate compound selected from the group consisting of alkali metal carbonates, alkaline earth metal carbonates, ammonium carbonate, alkali metal hydrogencarbonates, and ammonium hydrogencarbonate.

18. The method according to claim 14, wherein step (3) is a step of determining that the gastric acidity of the mammalian subject is the same as or higher than the gastric acidity of the control mammal when the measured ¹³CO₂ behavior and the reference ¹³CO₂ behavior are the same, or that the gastric acidity of the mammalian subject is lower than the gastric acidity of the control mammal when the measured ¹³CO₂ is lower than the reference ¹³CO₂.

19. The method according to claim 13 comprising as step (4) a step of determining enzyme activity (metabolic capacity) of CYP2C19 alone or both CYP2C19 and CYP3A4 in a mammalian subject, wherein step (4) is a step of determining that the enzyme activity (metabolic capacity) of CYP2C19 alone or both CYP2C19 and CYP3A4 in the mammalian subject is normal or high when step (3) determines that the gastric acidity of the mammalian subject is the same as or higher than the gastric acidity of the control mammal; or that the enzyme activity (metabolic capacity) of CYP2C19 alone or both CYP2C19 and CYP3A4 in the mammalian subject is low when step (3) determines that the gastric acidity of the mammalian subject is lower than the gastric acidity of the control mammal.

20. The method according to claim 18 comprising as step (4) a step of determining effect of a drug metabolized by CYP2C19 alone or both CYP2C19 and CYP3A4 on a mammalian subject, wherein
(a) in the case where the drug metabolized by CYP2C19 alone or both CYP2C19 and CYP3A4 shows an effect before being metabolized, step (4) is a step of determining that the effect of the drug on the mammalian subject is low when step (3) determines that the gastric acidity of the mammalian subject is the same as or higher than the gastric acidity of the control mammal, or that the effect of the drug on the mammalian subject is high when step (3) determines that the gastric acidity of the mammalian subject is lower than the gastric acidity of the control mammal; or
(b) in the case where the drug metabolized by CYP2C19 alone or both CYP2C19 and CYP3A4 shows an effect by metabolization, step (4) is a step of determining that the effect of the drug on the mammalian subject is high when step (3) determines that the gastric acidity of the mammalian subject is the same as or higher than the gastric acidity of the control mammal, or that the effect of the drug on the mammalian subject is low when step (3) determines that the gastric acidity of the mammalian subject is lower than the gastric acidity of the control mammal.

21. The method according to claim 18 comprising as step (4) a step of determining susceptibility of a mammalian subject to a drug metabolized by CYP2C19 alone or both CYP2C19 and CYP3A4, wherein
(a) in the case where the drug metabolized by CYP2C19 alone or both CYP2C19 and CYP3A4 shows an effect before being metabolized, step (4) is a step of determining that the susceptibility of the mammalian subject to the drug is high when step (3) determines that the gastric acidity of the mammalian subject is the same as or higher than the gastric acidity of the control mammal, or that the susceptibility of the mammalian subject to the drug is low when step (3) determines that the gastric acidity of the mammalian subject is lower than the gastric acidity of the control mammal; or
(b) in the case where the drug metabolized by CYP2C19 alone or both CYP2C19 and CYP3A4 shows an effect by metabolization, step (4) is a step of determining that the susceptibility of the mammalian subject to the drug is low when step (3) determines that the gastric acidity of the mammalian subject is the same as or higher than the gastric acidity of the control mammal, or that the susceptibility of the mammalian subject to the drug is high when step (3) determines that the gastric acidity of the mammalian subject is lower than the gastric acidity of the control mammal.
